SEQUENCE LISTING (69 sequences)

SEQ ID NO: 1 - protein, 116 aa, Artificial Sequence
  Synthetic Sequence_Sequence of heavy chain variable region 2H6 HCVR of murine monoclonal antibody 2H6
QVQLQQSGAELVRPGTSVKVSCKASGYAFSDYLIEWAKQRPGQGLEWIGVINPGSGGSNYNEKIKDRATLTADKSSSTAYMQLSSLTSEDSAVYFCARGGGGFTYWGQGTLVTVSA

SEQ ID NO: 2 - protein, 107 aa, Artificial Sequence
  Synthetic Sequence_Sequence of light chain variable region 2H6 LCVR of murine monoclonal antibody 2H6
EIQLTQTTSSLSASLGDRVTISCRASQDISNYLNWYQQKPDGTIKLLLNFASRLHSGVPSRFSGSGSGTDFFLTISNLEQDDIATYFCQQGSTLPWTFGGGTKLEIK

SEQ ID NO: 3 - protein, 10 aa, Artificial Sequence
  Synthetic Sequence_2H6 HCDR1
GYAFSDYLIE

SEQ ID NO: 4 - protein, 17 aa, Artificial Sequence
  Synthetic Sequence_2H6 HCDR2
VINPGSGGSNYNEKIKD

SEQ ID NO: 5 - protein, 7 aa, Artificial Sequence
  Synthetic Sequence_2H6 HCDR3
GGGGFTY

SEQ ID NO: 6 - protein, 11 aa, Artificial Sequence
  Synthetic Sequence_2H6 LCDR1
RASQDISNYLN

SEQ ID NO: 7 - protein, 7 aa, Artificial Sequence
  Synthetic Sequence_2H6 LCDR2
FASRLHS

SEQ ID NO: 8 - protein, 9 aa, Artificial Sequence
  Synthetic Sequence_2H6 LCDR3
QQGSTLPWT

SEQ ID NO: 9 - protein, 112 aa, Artificial Sequence
  Synthetic Sequence_Sequence of heavy chain variable region 9E5 HCVR of 9E5
QVQLQQPGADLVKPGASVKMSCKASGYILTTYWITWVKQRPGQGLEWIGDIHPGSGSTKYNEKFKSKATLTVDTSSSTAYMQLTRLSSEDSAVYYCARRDYWGQGTTLTVSS

SEQ ID NO: 10 - protein, 112 aa, Artificial Sequence
  Synthetic Sequence_Sequence of light chain variable region 9E5 LCVR of 9E5
DVLMTQSPLSLPVSLGDQASISCRSSQNIVNSQGNTYLEWYLQKPGESPKLLIYKVTNRFSGVPDRFSGSGSGTDFTLKISRVEAEDLGVYYCFQASLVPWTFGGGTKLEIK

SEQ ID NO: 11 - protein, 10 aa, Artificial Sequence
  Synthetic Sequence_9E5 HCDR1
GYILTTYWIT

SEQ ID NO: 12 - protein, 17 aa, Artificial Sequence
  Synthetic Sequence_9E5 HCDR2
DIHPGSGSTKYNEKFKS

SEQ ID NO: 13 - protein, 3 aa, Artificial Sequence
  Synthetic Sequence_9E5 HCDR3
RDY

SEQ ID NO: 14 - protein, 16 aa, Artificial Sequence
  Synthetic Sequence_9E5 LCDR1
RSSQNIVNSQGNTYLE

SEQ ID NO: 15 - protein, 7 aa, Artificial Sequence
  Synthetic Sequence_9E5 LCDR2
KVTNRFS

SEQ ID NO: 16 - protein, 9 aa, Artificial Sequence
  Synthetic Sequence_9E5 LCDR3
FQASLVPWT

SEQ ID NO: 17 - protein, 446 aa, Artificial Sequence
  Synthetic Sequence_hu2H6 HC
QVQLVQSGAEVKKPGSSVKVSCKASGYAFSDYLIEWVRQAPGQGLEWMGVINPGSGGSNYNEKIKDRVTLTADKSTSTAYMELSSLRSEDTAVYYCARGGGGFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 214 aa, Artificial Sequence
  Synthetic Sequence_hu2H6 LC
DIQLTQSPSSLSASVGDRVTITCRASQDISNYLNWYQQKPGKTIKLLLNFASRLHSGVPSRFSGSGSGTDFTFTISSLQPEDIATYYCQQGSTLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 442 aa, Artificial Sequence
  Synthetic Sequence_hu9E5 HC
QVQLVQSGAEVKKPGASVKVSCKASGYILTTYWITWVRQAPGQGLEWMGDIHPGSGSTKYNEKFKSRVTLTVDTSISTAYMELSRLRSEDTAVYYCARRDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 219 aa, Artificial Sequence
  Synthetic Sequence_hu9E5 LC
DIVMTQSPLSLPVTPGEPASISCRSSQNIVNSQGNTYLEWYLQKPGQSPQLLIYKVTNRFSGVPDRFSGSGSGTDFTLKISRVEAEDVGVYYCFQASLVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 98 aa, Artificial Sequence
  Synthetic Sequence_Preferable human germline heavy chain template IGHV1-69 for 2H6
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAR

SEQ ID NO: 22 - protein, 95 aa, Artificial Sequence
  Synthetic Sequence_Preferable human germline light chain template IGkV1-33 for 2H6
DIQMTQSPSSLSASVGDRVTITCQASQDISNYLNWYQQKPGKAPKLLIYDASNLETGVPSRFSGSGSGTDFTFTISSLQPEDIATYYCQQYDNLP

SEQ ID NO: 23 - protein, 98 aa, Artificial Sequence
  Synthetic Sequence_Preferable human germline heavy chain template IGHV1-2 for 9E5
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 24 - protein, 100 aa, Artificial Sequence
  Synthetic Sequence_Preferable human germline light chain template IGkV2-28 for 9E5
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTP

SEQ ID NO: 25 - protein, 116 aa, Artificial Sequence
  Synthetic Sequence_hu2H6-H1a
QVQLVQSGAEVKKPGSSVKVSCKASGGTFSDYLIEWVRQAPGQGLEWMGVINPGSGGSNYNEKIKDRVTITADKSTSTAYMELSSLRSEDTAVYYCARGGGGFTYWGQGTLVTVSS

SEQ ID NO: 26 - protein, 116 aa, Artificial Sequence
  Synthetic Sequence_hu2H6-H1b
QVQLVQSGAEVKKPGSSVKVSCKASGYAFSDYLIEWVRQAPGQGLEWMGVINPGSGGSNYNEKIKDRVTLTADKSTSTAYMELSSLRSEDTAVYYCARGGGGFTYWGQGTLVTVSS

SEQ ID NO: 27 - protein, 126 aa, Artificial Sequence
  Synthetic Sequence_hu2H6-H1c
QVQLVQSGAEVKKPGSSVKVSCKASGYAFSDYLIEWVRQAPGQGLEWIGVINPGSGGSNYNEKIKDRATLTADKSTSTAYMELSSLRSEDTAVYYCARGGGGFTYWGQGTLVTVSSFGQGTKLEIK

SEQ ID NO: 28 - protein, 112 aa, Artificial Sequence
  Synthetic Sequence_hu9E5-H1a
QVQLVQSGAEVKKPGASVKVSCKASGYTFTTYWITWVRQAPGQGLEWMGDIHPGSGSTKYNEKFKSRVTMTVDTSISTAYMELSRLRSEDTAVYYCARRDYWGQGTTVTVSS

SEQ ID NO: 29 - protein, 112 aa, Artificial Sequence
  Synthetic Sequence_hu9E5-H1b
QVQLVQSGAEVKKPGASVKVSCKASGYTFTTYWITWVRQAPGQGLEWMGDIHPGSGSTKYNEKFKSRVTLTVDTSISTAYMELSRLRSEDTAVYYCARRDYWGQGTTVTVSS

SEQ ID NO: 30 - protein, 112 aa, Artificial Sequence
  Synthetic Sequence_hu9E5-H1c
QVQLVQSGAEVKKPGASVKVSCKASGYILTTYWITWVRQAPGQGLEWMGDIHPGSGSTKYNEKFKSRVTLTVDTSISTAYMELSRLRSEDTAVYYCARRDYWGQGTTVTVSS

SEQ ID NO: 31 - protein, 107 aa, Artificial Sequence
  Synthetic Sequence_hu2H6-L1a
DIQMTQSPSSLSASVGDRVTITCRASQDISNYLNWYQQKPGKAPKLLLNFASRLHSGVPSRFSGSGSGTDFTFTISSLQPEDIATYYCQQGSTLPWTFGGGTKVEIK

SEQ ID NO: 32 - protein, 107 aa, Artificial Sequence
  Synthetic Sequence_hu2H6-L1b
DIQLTQSPSSLSASVGDRVTITCRASQDISNYLNWYQQKPGKAPKLLLNFASRLHSGVPSRFSGSGSGTDFTFTISSLQPEDIATYYCQQGSTLPWTFGGGTKVEIK

SEQ ID NO: 33 - protein, 107 aa, Artificial Sequence
  Synthetic Sequence_hu2H6-L1c
DIQLTQSPSSLSASVGDRVTITCRASQDISNYLNWYQQKPGKTIKLLLNFASRLHSGVPSRFSGSGSGTDFTFTISSLQPEDIATYYCQQGSTLPWTFGGGTKVEIK

SEQ ID NO: 34 - protein, 112 aa, Artificial Sequence
  Synthetic Sequence_hu9E5-L1a
DIVMTQSPLSLPVTPGEPASISCRSSQNIVNSQGNTYLEWYLQKPGQSPQLLIYKVTNRFSGVPDRFSGSGSGTDFTLKISRVEAEDVGVYYCFQASLVPWTFGGGTKVEIK

SEQ ID NO: 35 - protein, 112 aa, Artificial Sequence
  Synthetic Sequence_hu9E5-L1b
DVVMTQSPLSLPVTPGEPASISCRSSQNIVNSQGNTYLEWYLQKPGQSPQLLIYKVTNRFSGVPDRFSGSGSGTDFTLKISRVEAEDVGVYYCFQASLVPWTFGGGTKVEIK

SEQ ID NO: 36 - protein, 112 aa, Artificial Sequence
  Synthetic Sequence_hu9E5-L1c
DVLMTQSPLSLPVTPGEPASISCRSSQNIVNSQGNTYLEWYLQKPGQSPQLLIYKVTNRFSGVPDRFSGSGSGTDFTLKISRVEAEDVGVYYCFQASLVPWTFGGGTKVEIK

SEQ ID NO: 37 - protein, 116 aa, Artificial Sequence
  Synthetic Sequence_Sequence of heavy chain variable region 1D9 HCVR of 1D9
QVRLQQSGAELVRPGTSMRVSCKASGYAFTNYLINWVKQRPGQGLEWIGILNPGSGGTNYNENFKDKATLTADKSSNTAYMQLSSLTSEDSAVYFCIRGSPGFAYWGQGTLVTVSA

SEQ ID NO: 38 - protein, 107 aa, Artificial Sequence
  Synthetic Sequence_Sequence of light chain variable region 1D9 LCVR of 1D9
DIQMTQTTSSLSASLGDRVTISCRASQDINIYLNWYQQKPDGTVKLLIYSTSGLHSGVPSRFNGSGSGTDYSLTISNLEQEDIATYFCQQGYTLPYTFGGGTKLEIK

SEQ ID NO: 39 - protein, 10 aa, Artificial Sequence
  Synthetic Sequence_1D9 HCDR1
GYAFTNYLIN

SEQ ID NO: 40 - protein, 17 aa, Artificial Sequence
  Synthetic Sequence_1D9 HCDR2
ILNPGSGGTNYNENFKD

SEQ ID NO: 41 - protein, 7 aa, Artificial Sequence
  Synthetic Sequence_1D9 HCDR3
GSPGFAY

SEQ ID NO: 42 - protein, 11 aa, Artificial Sequence
  Synthetic Sequence_1D9 LCDR1
RASQDINIYLN

SEQ ID NO: 43 - protein, 7 aa, Artificial Sequence
  Synthetic Sequence_1D9 LCDR2
STSGLHS

SEQ ID NO: 44 - protein, 9 aa, Artificial Sequence
  Synthetic Sequence_1D9 LCDR3
QQGYTLPYT

SEQ ID NO: 45 - protein, 116 aa, Artificial Sequence
  Synthetic Sequence_Sequence of heavy chain variable region 14C10 HCVR of 14C10
QVQVQQSGAELVRPGTSVKVSCKASGYAFTNYLIEWVKQRPGQGLEWIGVINPEFGGTNYNEKFKGKATLTADKSSSTAYMQLSSLTSEDSAVYFCARGGGGFTYWGQGTLVTVSA

SEQ ID NO: 46 - protein, 107 aa, Artificial Sequence
  Synthetic Sequence_Sequence of light chain variable region14C10 LCVR of 14C10
HIQMTQTTSSLSASLGDRVTISCRASQDISSHLNWYQQKPDGTVKLLISYTSRLHSGVPSRFSGSGSGADYSLTISNLEQEDIATYFCQQGNTLPWTFGGGTKLEIK

SEQ ID NO: 47 - protein, 10 aa, Artificial Sequence
  Synthetic Sequence_14C10 HCDR1
GYAFTNYLIE

SEQ ID NO: 48 - protein, 17 aa, Artificial Sequence
  Synthetic Sequence_14C10 HCDR2
VINPEFGGTNYNEKFKG

SEQ ID NO: 49 - protein, 7 aa, Artificial Sequence
  Synthetic Sequence_14C10 HCDR3
GGGGFTY

SEQ ID NO: 50 - protein, 11 aa, Artificial Sequence
  Synthetic Sequence_14C10 LCDR1
RASQDISSHLN

SEQ ID NO: 51 - protein, 7 aa, Artificial Sequence
  Synthetic Sequence_14C10 LCDR2
YTSRLHS

SEQ ID NO: 52 - protein, 9 aa, Artificial Sequence
  Synthetic Sequence_14C10 LCDR3
QQGNTLPWT

SEQ ID NO: 53 - protein, 119 aa, Artificial Sequence
  Synthetic Sequence_Sequence of heavy chain variable region 38B4 HCVR of 38B4
QVRLKQSGAELVRPGASVKVSCKASGYTFTDYYINWVKQRPGQGLEWIAGIYPGTGNTYYNEKFKGKATLTAERSSSTAYMQLTSLTSEDSAVYFCTRRGLPSLCFDYWGQGTTLTVSS

SEQ ID NO: 54 - protein, 107 aa, Artificial Sequence
  Synthetic Sequence_Sequence of light chain variable region 38B4 LCVR of 38B4
DFQMTQTTSSLSASLGDRVTISCSASQGISNYLNWYQQKPDGTVKLLIYYTSSLHSGVPSRFSGSGSGTDYSLTISNLEPEDIATYYCQQYSKLPPTFGGGTKLEIK

SEQ ID NO: 55 - protein, 10 aa, Artificial Sequence
  Synthetic Sequence_38B4 HCDR1
GYTFTDYYIN

SEQ ID NO: 56 - protein, 17 aa, Artificial Sequence
  Synthetic Sequence_38B4 HCDR2
GIYPGTGNTYYNEKFKG

SEQ ID NO: 57 - protein, 10 aa, Artificial Sequence
  Synthetic Sequence_38B4 HCDR3
RGLPSLCFDY

SEQ ID NO: 58 - protein, 11 aa, Artificial Sequence
  Synthetic Sequence_38B4 LCDR1
SASQGISNYLN

SEQ ID NO: 59 - protein, 7 aa, Artificial Sequence
  Synthetic Sequence_38B4 LCDR2
YTSSLHS

SEQ ID NO: 60 - protein, 9 aa, Artificial Sequence
  Synthetic Sequence_38B4 LCDR3
QQYSKLPPT

SEQ ID NO: 61 - protein, 446 aa, Artificial Sequence
  Synthetic Sequence_hu2H6-M HC
QVQLVQSGAEVKKPGSSVKVSCKASGYAFSDYLIEWVRQAPGQGLEWMGVINPGSGGSNYNEKIKDRVTLTADKSTSTAYMELSSLRSEDTAVYYCARGGGGFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 446 aa, Artificial Sequence
  Synthetic Sequence_hu2H6-SELFNS HC
QVQLVQSGAEVKKPGSSVKVSCKASGYAFSDYLIEWVRQAPGQGLEWMGVINPGSGGSNYNEKIKDRVTLTADKSTSTAYMELSSLRSEDTAVYYCARGGGGFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 442 aa, Artificial Sequence
  Synthetic Sequence_hu9E5-M HC
QVQLVQSGAEVKKPGASVKVSCKASGYILTTYWITWVRQAPGQGLEWMGDIHPGSGSTKYNEKFKSRVTLTVDTSISTAYMELSRLRSEDTAVYYCARRDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 442 aa, Artificial Sequence
  Synthetic Sequence_hu9E5-SELF HC
QVQLVQSGAEVKKPGASVKVSCKASGYILTTYWITWVRQAPGQGLEWMGDIHPGSGSTKYNEKFKSRVTLTVDTSISTAYMELSRLRSEDTAVYYCARRDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 450 aa, Artificial Sequence
  Synthetic Sequence_005M-HC
QVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 215 aa, Artificial Sequence
  Synthetic Sequence_005M-LC
DIQMTQSPSSLSASVGDRVTIKCQASQSISSRLAWYQQKPGKPPKLLIYRASTLASGVPSRFSGSGSGTDFTLTISSLQPEDVATYYCQCTGYGISWPIGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 442 aa, Artificial Sequence
  Synthetic Sequence_hu9E5-SELFNS HC
QVQLVQSGAEVKKPGASVKVSCKASGYILTTYWITWVRQAPGQGLEWMGDIHPGSGSTKYNEKFKSRVTLTVDTSISTAYMELSRLRSEDTAVYYCARRDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 450 aa, Artificial Sequence
  Synthetic Sequence_APX005-SELF HC
QVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 450 aa, Artificial Sequence
  Synthetic Sequence_APX005-SELFNS HC
QVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2022/0025060 A1
(43) Pub. Date: Jan. 27, 2022

(54) ANTI-CD40 ANTIBODY, ANTIGEN BINDING FRAGMENTAND PHARMACEUTICAL USE THEREOF

(71) Applicants: JIANGSU HENGRUI MEDICINE CO., LTD., Lianyungang (CN); SHANGHAI HENGRUI PHARMACEUTICAL CO., LTD., Shanghai (CN)

(72) Inventors: Cheng LIAO, Lianyungang (CN); Jiahua JIANG, Lianyungang (CN); Zupeng XU, Lianyungang (CN); Lianshan ZHANG, Lianyungang (CN); Yuan LIN, Lianyungang (CN); Kan LIN, Lianyungang (CN); Xueming QIAN, Lianyungang (CN); Fei TENG, Lianyungang (CN)

(21) Appl. No.: 17/296,519

(22) PCT Filed: Nov. 29, 2019
(86) PCT No.: PCT/CN2019/121941

§ 371 Date: May 24, 2021

(30) Foreign Application Priority Data
Nov. 30, 2018 (CN) 201811448228.1

(51) Int. Cl. C07K 16/28; C12N 15/63; A61P 35/00

(52) U.S. Cl. CPC C07K 16/2878; C12N 15/63; A61P 35/00; A61K 2039/505; C07K 2317/51; C07K 2317/24; C07K 2317/515

(57) ABSTRACT

The present invention relates to an anti-CD40 antibody, an antigen binding fragment and a pharmaceutical use thereof. Heavy-chain constant regions of the anti-CD40 antibody and the antigen binding fragment thereof contain mutations. Due to the mutations, the anti-CD40 antibody loses the binding activity to FcγRIII, and the binding of the anti-CD40 antibody and FcγRIIB is enhanced, thereby losing the antibody-dependent cytotoxicity (ADCC) but improving FcγRIIB-mediated antibody crosslinking. The mutations in the heavy-chain constant regions enhance the activation of CD40, and enhance the presentation of dendritic cells to antigens. The anti-CD40 antibody and the antigen binding fragment thereof can be used as anti-cancer drugs to treat CD40-mediated diseases or symptoms.

The present application claims the priority of Chinese patent application “anti-CD40 antibody, antigen-binding fragment and pharmaceutical use thereof” (application number CN201811448228.1) filed on Nov. 30, 2018.

### FIELD OF THE INVENTION

The present disclosure relates to an anti-CD40 antibody or antigen-binding fragment thereof comprising mutation(s) in the heavy chain constant region, a chimeric antibody or a humanized antibody comprising CDRs of the anti-CD40 antibody, and a pharmaceutical composition comprising the anti-human CD40 antibody or antigen-binding fragment thereof, and the use of the same as an anticancer agent.

### BACKGROUND OF THE INVENTION

Cancers have become the biggest health challenge faced by human society for a long time. Traditional therapies such as surgery, chemotherapy and radiotherapy show little effect in the treatment of disseminated solid tumors. Tumor immunotherapy is a hot spot in the field of tumor therapy, and tumor immunotherapy by T cells is at a core position. Tumor immunotherapy makes full use of killer T cells, and mobilizes the killer T cells in tumor patients to kill the tumor. Tumor immunotherapy may be one of the most effective and safest ways to treat tumors. Tumor immunotherapy currently has favorable prospects for the treatment of several different types of cancers, including disseminated metastatic tumors.

The activation of T cells in the human body adopts a dual-signal pathway system: MI-IC-antigen peptides are presented to T cells through antigen presenting cells (APC) to provide the first signal; a series of co-stimulatory molecules are required to provide the second signal, and then T cells produce a normal immune response. This dual-signal pathway system plays a vital role in the balance of the immune system in vivo. It strictly regulates the body's different immune responses to self-antigens and non-self-antigens. In the absence of the second signal provided by the co-stimulatory molecule, T cells will not be responsive or generate a sustained specific immune response, consequently resulting in tolerance. Therefore, the second signal pathway plays a very critical regulatory role in the entire process of the body's immune response.

CD40 is one of the glycoproteins expressed on the cell surface. It is a type I intra-membrane glycoprotein with a molecular weight of about 48 kDa. CD40 belongs to the tumor necrosis factor receptor (TNFR) superfamily and plays an important role in the immune system. CD40 is expressed in a variety of immune cells, such as B cells, dendritic cells, monocytes and macrophages. When signal transduction occurs through CD40, specialized antigen-presenting cells are activated. The natural ligand of CD40 is named as CD154 or CD40L, and it is known to be expressed mainly on mature T lymphocytes. CD40L-mediated signal transduction can trigger some cellular biological events, including immune cell activation, proliferation, and the production of cytokines and chemokines. CD40 signaling is very important for T cell-dependent immune responses, especially in the context of tumor environment. CD40-stimulated dendritic cells can activate tumor-specific effector T cells, which have the potential to eradicate tumor cells.

The expression of CD40 can be found in many normal cells and tumor cells including B lymphocytes. For example, melanoma is a tumor that expresses CD40, and 30% to 70% of solid tumors also exhibit CD40 expression. At present, it is known that the activation of CD40 can effectively trigger anti-tumor responses (Tong et al., Cancer Gene Therapy, 2003, 10: 1-13), including immune activation of tumor-specific T cell responses, direct effect on the apoptosis of CD40-positive tumors, and stimulation-mediated humoral response of ADCC. It has been observed that the eradication of tumor is strongly correlated with the presence of tumor-specific cytotoxic T lymphocytes. At the same time, it should be noticed that systemic administration of CD40-antibody is associated with side effects, such as shock syndrome and cytokine release syndrome (van Mierlo et al., Proc. Natl. Acad. Sci. USA, 2002, 99: 5561-5566).

At present, many international pharmaceutical companies are developing monoclonal antibodies against CD40 as mentioned above, which specifically stimulate immune activation to maximize the patient's own immune system to respond to tumors, so as to achieve the purpose of killing tumor cells. Related patents involve such as PCT/CN2018/089252, CN1198647, CN1369015, CN1582165, CN100430419, CN101014386, CN101237882, CN101289510, CN101490086, CN103842382, CN104918957, WO2002028904, WO2011123489, WO2012149356, WO2013034904, WO201509853, WO2016196314, WO2017040932, WO2017004006, etc. So far, anti-CD40 antibodies available from Pfizer (related products have been licensed to Roche), Alligator and other companies have been observed having favorable tumor killing effects in preclinical animal models, and have entered Phase I clinical trials.

As for mutations in antibody constant region, WO2006019447, WO2014145806, U.S. Pat. Nos. 8,734,791, 9,657,106, 8,084,582, WO2008150494, WO2004099249 disclose mutations of S267E, L328F, and N325S of the antibody heavy chain. The mutations delete the binding ability of the antibody to FcγRIII, whereas enhance the binding ability to FcγRIIB. Therefore, the antibody-dependent cellular cytotoxicity (ADCC) is deleted, while FcγRIIB-mediated cross-linking is enhanced, thereby enhancing the activation of CD40 and enhancing the antigen-presentation by dendritic cells.

The present disclosure aims to provide an anti-CD40 antibody with high affinity, high selectivity, and high biological activity, which lacks of antibody-dependent cellular cytotoxicity (ADCC), but has enhanced FcγRIIB-mediated crosslinking, thereby inhibiting in vivo tumor growth. The antibodies of the present disclosure can be used as medicament or used in a composition for the treatment of cancers mediated by CD40 and mediated by CD40 pathway.

### SUMMARY OF THE INVENTION

The present disclosure provides a CD40 antibody or antigen-binding fragment thereof, which comprises:

a light chain variable region of the antibody, comprising at least one LCDR shown as a sequence selected from the group consisting of: SEQ ID NO: 6, SEQ ID NO: 7, SEQ ID NO: 8; SEQ ID NO: 14, SEQ ID NO: 15, SEQ ID NO: 16; SEQ ID NO: 42, SEQ ID NO: 43, SEQ ID NO: 44; SEQ ID NO: 50, SEQ ID NO: 51, SEQ ID NO: 52; SEQ ID NO: 58, SEQ ID NO: 59 and SEQ ID NO: 60; and/or

a heavy chain variable region of the antibody, comprising at least one HCDR shown as a sequence selected from the group consisting of: SEQ ID NO: 3, SEQ ID NO: 4, SEQ ID NO: 5, SEQ ID NO: 11, SEQ ID NO: 12, SEQ ID NO: 13; SEQ ID NO: 39, SEQ ID NO: 40, SEQ ID NO: 41; SEQ ID NO: 47, SEQ ID NO: 48, SEQ ID NO:49; SEQ ID NO:55, SEQ ID NO:56 and SEQ ID NO:57.

In some embodiments, the anti-CD40 antibody or antigen-binding fragment thereof as described above, comprises a light chain variable region comprising LCDR1 as shown in SEQ ID NO: 6, SEQ ID NO: 14, SEQ ID NO: 42, SEQ ID NO: 50, or SEQ ID NO: 58.

In some embodiments, the anti-CD40 antibody or antigen-binding fragment thereof as described above, comprises a light chain variable region of the antibody comprising LCDR2 as shown in SEQ ID NO: 7, SEQ ID NO: 15, SEQ ID NO: 43, SEQ ID NO: 51 or SEQ ID NO: 59.

In some embodiments, the anti-CD40 antibody or antigen-binding fragment thereof as described above, comprises a light chain variable region of the antibody comprising LCDR3 as shown in SEQ ID NO: 8, SEQ ID NO: 16, SEQ ID NO: 44, SEQ ID NO: 52 or SEQ ID NO: 60.

In some embodiments, the anti-CD40 antibody or antigen-binding fragment thereof as described above, comprises a heavy chain variable region of the antibody comprising HCDR1 as shown in SEQ ID NO: 3, SEQ ID NO: 11, SEQ ID NO: 39, SEQ ID NO: 47 or SEQ ID NO: 55.

In some embodiments, the anti-CD40 antibody or antigen-binding fragment thereof as described above, comprises a heavy chain variable region of the antibody comprising HCDR2 as shown in SEQ ID NO: 4, SEQ ID NO: 12, SEQ ID NO: 40, SEQ ID NO: 48 or SEQ ID NO: 56.

In some embodiments, the anti-CD40 antibody or antigen-binding fragment thereof as described above, comprises a heavy chain variable region of the antibody comprising HCDR3 as shown in SEQ ID NO: 5, SEQ ID NO: 13, SEQ ID NO: 41, SEQ ID NO: 49 or SEQ ID NO: 57.

In some embodiments, the anti-CD40 antibody or antigen-binding fragment thereof as described above, comprises a light chain variable region of the antibody comprising LCDR1, LCDR2 and LCDR3 as shown in SEQ ID NO: 6, SEQ ID NO: 7 and SEQ ID NO: 8, respectively.

In some embodiments, the anti-CD40 antibody or antigen-binding fragment thereof as described above, comprises a light chain variable region of the antibody comprising LCDR1, LCDR2 and LCDR3 as shown in SEQ ID NO: 14, SEQ ID NO: 15 and SEQ ID NO: 16, respectively.

In some embodiments, the anti-CD40 antibody or antigen-binding fragment thereof as described above, comprises a light chain variable region of the antibody comprising LCDR1, LCDR2 and LCDR3 as shown in SEQ ID NO: 42, SEQ ID NO: 43 and SEQ ID NO: 44, respectively.

In some embodiments, the anti-CD40 antibody or antigen-binding fragment thereof as described above, comprises a light chain variable region of the antibody comprising LCDR1, LCDR2 and LCDR3 as shown in SEQ ID NO: 50, SEQ ID NO: 51 and SEQ ID NO: 52, respectively.

In some embodiments, the anti-CD40 antibody or antigen-binding fragment thereof as described above, comprises a light chain variable region of the antibody comprising LCDR1, LCDR2 and LCDR3 as shown in SEQ ID NO: 58, SEQ ID NO: 59 and SEQ ID NO: 60, respectively.

In some embodiments, the anti-CD40 antibody or antigen-binding fragment thereof as described above, comprises a heavy chain variable region of the antibody comprising HCDR1, HCDR2 and HCDR3 as shown in SEQ ID NO: 3, SEQ ID NO: 4 and SEQ ID NO: 5, respectively.

In some embodiments,the anti-CD40 antibody or antigen-binding fragment thereof as described above, comprises a heavy chain variable region of the antibody comprising HCDR1, HCDR2 and HCDR3 as shown in SEQ ID NO: 11, SEQ ID NO: 12 and SEQ ID NO: 13, respectively.

In some embodiments, the anti-CD40 antibody or antigen-binding fragment thereof as described above, comprises a heavy chain variable region of the antibody comprising HCDR1, HCDR2 and HCDR3 as shown in SEQ ID NO: 39, SEQ ID NO: 40 and SEQ ID NO: 41, respectively.

In some embodiments, the anti-CD40 antibody or antigen-binding fragment thereof as described above, comprises a heavy chain variable region of the antibody comprising HCDR1, HCDR2 and HCDR3 as shown in SEQ ID NO: 47, SEQ ID NO: 48 and SEQ ID NO: 49, respectively.

In some embodiments, the anti-CD40 antibody or antigen-binding fragment thereof as described above, comprises a heavy chain variable region of the antibody comprising HCDR1, HCDR2 and HCDR3 as shown in SEQ ID NO: 55, SEQ ID NO: 56 and SEQ ID NO: 57, respectively.

In some particular embodiments, the anti-CD40 antibody or antigen-binding fragment thereofcomprises a light chain variable region of the antibody comprising:

LCDR1, LCDR2 and LCDR3 as shown in SEQ ID NO: 6, SEQ ID NO: 7 and SEQ ID NO: 8, respectively; or

LCDR1, LCDR2 and LCDR3 as shown in SEQ ID NO: 14, SEQ ID NO: 15 and SEQ ID NO: 16, respectively; or

LCDR1, LCDR2 and LCDR3 as shown in SEQ ID NO: 42, SEQ ID NO: 43 and SEQ ID NO: 44, respectively; or

LCDR1, LCDR2 and LCDR3 as shown in SEQ ID NO: 50, SEQ ID NO: 51 and SEQ ID NO: 52, respectively; or

LCDR1, LCDR2 and LCDR3 as shown in SEQ ID NO: 58, SEQ ID NO: 59 and SEQ ID NO: 60, respectively; and,

a heavy chain variable region of the antibody comprising:

HCDR1, HCDR2 and HCDR3 as shown in SEQ ID NO: 3, SEQ ID NO: 4 and SEQ ID NO: 5, respectively; or

HCDR1, HCDR2 and HCDR3 as shown in SEQ ID NO: 11, SEQ ID NO: 12 and SEQ ID NO: 13, respectively; or

HCDR1, HCDR2 and HCDR3 as shown in SEQ ID NO: 39, SEQ ID NO: 40 and SEQ ID NO: 41, respectively; or

HCDR1, HCDR2 and HCDR3 as shown in SEQ ID NO: 47, SEQ ID NO: 48 and SEQ ID NO: 49, respectively; or

HCDR1, HCDR2 and HCDR3 as shown in SEQ ID NO: 55, SEQ ID NO: 56 and SEQ ID NO: 57, respectively.

In some particular embodiments, the anti-CD40 antibody or antigen-binding fragment thereof can be any one selected from:

(1) the light chain variable region of the antibody comprises LCDR1, LCDR2 and LCDR3 as shown in SEQ ID NO: 6, SEQ ID NO: 7 and SEQ ID NO: 8, respectively; the heavy chain variable region of the antibody comprises HCDR1, HCDR2 and HCDR3 as shown in SEQ ID NO: 3, SEQ ID NO: 4 and SEQ ID NO: 5, respectively;

(2) the light chain variable region of the antibody comprises LCDR1, LCDR2 and LCDR3 as shown in SEQ ID NO: 14, SEQ ID NO: 15 and SEQ ID NO: 16, respectively; the heavy chain variable region of the antibody comprises HCDR1, HCDR2 and HCDR3 as shown in SEQ ID NO: 11, SEQ ID NO: 12 and SEQ ID NO:13, respectively;

(3) the light chain variable region of the antibody comprises LCDR1, LCDR2 and LCDR3 as shown in SEQ ID NO: 42, SEQ ID NO: 43 and SEQ ID NO: 44, respectively; the heavy chain variable region of the antibody comprises HCDR1, HCDR2 and HCDR3 as shown in SEQ ID NO: 39, SEQ ID NO:40 and SEQ ID NO:41, respectively;

(4) the light chain variable region of the antibody comprises LCDR1, LCDR2 and LCDR3 as shown in SEQ ID NO: 50, SEQ ID NO: 51 and SEQ ID NO: 52, respectively; the heavy chain variable region of the antibody comprises HCDR1, HCDR2 and HCDR3 as shown in SEQ ID NO: 47, SEQ ID NO:48 and SEQ ID NO:49, respectively; and

(5) the light chain variable region of the antibody comprises LCDR1, LCDR2 and LCDR3 as shown in SEQ ID NO: 58, SEQ ID NO: 59 and SEQ ID NO: 60, respectively; the heavy chain variable region of the antibody comprises HCDR1, HCDR2 and HCDR3 as shown in SEQ ID NO: 55, SEQ ID NO:56 and SEQ ID NO:57, respectively.

In some particular embodiments, the light chain variable region sequence of the antibody is selected from the group consisting of SEQ ID NO: 2 or SEQ ID NO: 10; the heavy chain variable region sequence is selected from SEQ ID NO: 1 or SEQ ID NO: 9.

The anti-CD40 antibody or antigen-binding fragment thereof described above can be a murine antibody or a chimeric antibody.

In some particular embodiments, the amino acid sequence of the heavy chain variable region of the murine antibody or the chimeric antibody is as shown in SEQ ID NO: 1, and the amino acid sequence of the light chain variable region is as shown in SEQ ID NO: 2.

In other particular embodiments, the amino acid sequence of the heavy chain variable region of the murine antibody or the chimeric antibody is as shown in SEQ ID NO: 9, and the amino acid sequence of the light chain variable region is as shown in SEQ ID NO: 10.

In other particular embodiments, the light chain variable region LCVR of the antibody or the antigen fragment is as shown in sequence SEQ ID NO: 38, and the heavy chain variable region HCVR is as shown in sequence SEQ ID NO: 37.

In other particular embodiments, the light chain variable region LCVR of the antibody or the antigen fragment is as shown in sequence SEQ ID NO: 46, and the heavy chain variable region HCVR is as shown in sequence SEQ ID NO: 45.

In other particular embodiments, the light chain variable region LCVR of the antibody or the antigen fragment is as shown in sequence SEQ ID NO: 54, and the heavy chain variable region HCVR is as shown in sequence SEQ ID NO: 53.

In some particular embodiments, the anti-CD40 antibody or antigen-binding fragment thereof is a murine antibody, a chimeric antibody, a humanized antibody, a human antibody or fragment(s) thereof.

In some particular embodiments, when the anti-CD40 antibody or antigen-binding fragment thereof is a murine antibody or fragment thereof, the light chain variable region of the antibody further comprises light chain FR region(s) or light chain constant region(s) of murine κ, λ chain or variant(s) thereof; and/or the heavy chain variable region of the antibody further comprises the heavy chain FR region(s) or heavy chain constant region(s) of murine IgG1, IgG2, IgG3, IgG4 or variant(s) thereof.

In some particular embodiments, when the anti-CD40 antibody or antigen-binding fragment thereof is a chimeric antibody or fragment thereof, it comprises the light chain constant region(s) of human κ, λ chain or variant(s) thereof, and/or comprises the heavy chain constant region(s) of human IgG1, IgG2, IgG3 or IgG4 or variant(s) thereof. In some particular embodiments, the light chain variable region sequence is as shown in SEQ ID NO: 2 or SEQ ID NO: 10, and/or the heavy chain variable region sequence is as shown in SEQ ID NO: 1 or SEQ ID NO: 9.

In some particular embodiments, when the anti-CD40 antibody or antigen-binding fragment thereof is a humanized antibody or fragment thereof, the light chain sequence of the antibody is: SEQ ID NO: 18 or SEQ ID NO: 20 or variant thereof; in particular, the variant has 0-10 amino acid change(s) in the light chain, more specifically, has amino acid mutation(s) at positions 2 and 3. The amino acids after mutation at positions 2 and 3 are each independently selected from I, V or L; the heavy chain sequence of the antibody is: SEQ ID NO: 17 or SEQ ID NO: 19 or variant thereof; the variant has 0-10 amino acid change(s) in the heavy chain, more specifically, has amino acid mutation(s) at positions 6 and 8. The amino acids after mutation are each independently selected from I, A or L.

In some particular embodiments of the anti-CD40 humanized antibody or fragment thereof as described above, the heavy chain variable region of the humanized antibody further comprises heavy chain constant region(s) or FR region(s) of human IgG1, IgG2, IgG3, IgG4 or variant(s) thereof, in particular comprises heavy chain constant region(s) or FR region(s) of human IgG1, IgG2 or IgG4, in particular comprises heavy chain constant region(s) or FR region(s) of human IgG1 or IgG2; and/or comprises light chain FR region(s) of human κ, λ chain or variant(s) thereof.

In some particular embodiments of the anti-CD40 humanized antibody or fragment thereof as described above, the light chain FR region sequence on the light chain variable region of the humanized antibody is derived from, for example, a human germline light chain IGkV1-33 as shown in sequence SEQ ID NO: 22; or derived from a human germline light chain IGkV2-28 as shown in sequence SEQ ID NO: 24.

In some particular embodiments of the anti-CD40 humanized antibody or fragment thereof as described above, the light chain variable region variant of the humanized antibody particularly has 0-10 amino acid change(s) in the light chain variable region; more particularly, has amino acid mutation(s) at positions 2 and 3; in particular, the amino acids after mutation are I, V or L.

In some particular embodiments, the anti-CD40 humanized antibody or the fragment thereof as described above further comprises a light chain constant region of a human kappa, lambda chain or variant thereof.

In some particular embodiments of the anti-CD40 humanized antibody or fragment thereof as described above, the heavy chain FR region sequence on the heavy chain variable region of the humanized antibody is derived from, for example, a human germline heavy chain IGHV1-69 as shown in sequence SEQ ID NO: 21, and/or derived from a human germline heavy chain IGkV1-33 as shown in sequence SEQ ID NO: 22; derived from a human germline heavy chain IGHV1-2 as shown in sequence SEQ ID NO: 23, and/or derived from a human germline heavy chain IGkV2-28 as shown in sequence SEQ ID NO: 24.

In some particular embodiments of the anti-CD40 humanized antibody or fragment thereof, the heavy chain variable region is selected from sequence as shown in one of SEQ ID NOs: 25-30 or variant thereof, and the light chain variable region is selected from sequence as shown in one of SEQ ID NOs:31-36 or variant thereof.

In some particular embodiments of the anti-CD40 humanized antibody or fragment thereof, the heavy chain variable region is as shown in SEQ ID NO: 26 or variant thereof, and the light chain variable region is as shown in SEQ ID NO: 33 or variant thereof.

In some particular embodiments, the heavy chain variable region is as shown in SEQ ID NO: 30 or variant thereof, and the light chain variable region is as shown in sequence SEQ ID NO: 34 or variant thereof.

In some particular embodiments, the heavy chain of the humanized anti-CD40 antibody is as shown in SEQ ID NO: 17, and the light chain is as shown in SEQ ID NO: 18.

In some particular embodiments, the heavy chain is as shown in SEQ ID NO: 19, and the light chain is as shown in SEQ ID NO: 20.

In some particular embodiments of the anti-CD40 humanized antibody or fragment thereof, the humanized antibody heavy chain sequence is as shown in SEQ ID NO: 61, 62, 63, 64 or 67 or variant thereof, and/or the light chain variable region is as shown in SEQ ID NO: 18, 20 or variant thereof.

In some particular embodiments, the heavy chain sequence of the anti-CD40 humanized antibody or the fragment thereof is the sequence as shown in SEQ ID NO: 61 or 62 or variant thereof, and the light chain sequence is the sequence as shown in SEQ ID NO: 18 or variant thereof; the heavy chain sequence is the sequence as shown in SEQ ID NO: 63, 64 or 67 or variant thereof, and the light chain sequence is the sequence as shown in SEQ ID NO: 20 or variant thereof.

The variant has 0-10 amino acid change(s) in the heavy chain variable region, in particular has amino acid mutations at positions 6 and 8, and in particular the amino acids after mutation are I, A or L.

Herein, the sequence shown in SEQ ID NO: 61 comprises an amino acid residue mutated into glutamic acid (E) on position 266 corresponding to SEQ ID NO: 17 (e.g. S266E);

the sequence shown in SEQ ID NO: 62 comprises an amino acid residue mutated into glutamic acid (E) on position 266 corresponding to SEQ ID NO: 17 (e.g. S266E), an amino acid residue mutated into serine (S) on position 324 corresponding to SEQ ID NO: 17 (e.g., N324S), and an amino acid residue mutated into phenylalanine (F) on position 327 corresponding to SEQ ID NO: 17 (e.g., L327F);

the sequence shown in SEQ ID NO: 63 comprises an amino acid residue mutated into glutamic acid (E) on position 262 corresponding to SEQ ID NO: 19 (e.g. S262E);

the sequence shown in SEQ ID NO: 64 comprises an amino acid residue mutated into glutamic acid (E) on position 262 corresponding to SEQ ID NO: 19 (e.g. S262E), and an amino acid residue mutated into phenylalanine (F) on position 323 corresponding to SEQ ID NO: 19 (for example, L323F);

the sequence shown in SEQ ID NO: 67 comprises an amino acid residue mutated into glutamic acid (E) on position 262 corresponding to SEQ ID NO: 19 (e.g. S262E); an amino acid residue mutated into serine (S) on position 320 corresponding to SEQ ID NO: 19 (e.g., N320S), and an amino acid residue mutated into phenylalanine (F) on position 323 corresponding to SEQ ID NO: 19 (e.g., L323F). Among them, the numbering of amino acid position is in accordance with the natural order. In some embodiments, the amino acid at the last position (such as lysine) of the heavy chain amino acid sequence of the anti-CD40 antibody or antigen-binding fragment thereof described above is mutated into alanine (A).

In some particular embodiments, the amino acid at the last position of the heavy chain sequence as shown in SEQ ID NO: 61, 62, 63, 64, or 67 is mutated into A.

In other particular embodiments, an antibody is provided, which comprises a heavy chain as shown in SEQ ID NO: 69 and a light chain as shown in SEQ ID NO: 66.

In other particular embodiments, an antibody is provided, which comprises a heavy chain as shown in SEQ ID NO: 68 and a light chain as shown in SEQ ID NO: 66.

In some particular embodiments of the anti-CD40 antibody or antigen-binding fragment thereof as described above, the antigen-binding fragment is Fab, Fv, sFv, F(ab′)₂, linear antibody, single-chain antibody, nanobody, domain antibody or multispecific antibody.

The present disclosure further provides a single-chain antibody, which comprises the heavy chain variable region and the light chain variable region of the anti-CD40 antibody or antigen-binding fragment thereof as described above.

The present disclosure further provides a multispecific antibody, which comprises the heavy chain variable region and the light chain variable region of the anti-CD40 antibody or antigen-binding fragment thereof as described above.

The present disclosure further provides a nucleic acid molecule (DNA or RNA) that encodes the anti-CD40 antibody or antigen-binding fragment thereof, multispecific antibody or single-chain antibody as described above.

The present disclosure further provides an expression vector comprising the nucleic acid molecule as described above.

The present disclosure further provides a host cell, which comprises or is transformed with the expression vector as described above. In some particular embodiments, the host cell is bacterium, yeast or mammalian cell, in particular Escherichia coli, Pichia pastoris, Chinese hamster ovary (CHO) cell or human embryonic kidney (HEK) 293 cell.

The present disclosure further provides an antibody-drug conjugate comprising the anti-CD40 antibody light chain variable region and/or heavy chain variable region as described above. The antibody-drug conjugate is well-known in the art, and is formed by connecting antibody, linker and drug. The known linkers involve cleavable linkers and non-cleavable linkers. For example, linkers involve but are not limited to SMCC, SPDP and the like. Drugs are also well-known in the art, such as DM1, DM4, MMAE, MMAF, etc.

The present disclosure further provides a pharmaceutical composition, which comprises the anti-CD40 antibody or antigen-binding fragment thereof, multispecific antibody or single-chain antibody, and pharmaceutically acceptable excipient(s), diluent(s) or carrier(s).

In some embodiments, the unit dose of the pharmaceutical composition may comprise 0.01% to 99% (by weight) of the anti-CD40 antibody or the fragment thereof, or the amount of the CD40 antibody or the fragment thereof in unit dose of the pharmaceutical composition(s) is from 0.1 mg to 2000 mg; in some embodiments, from 1 mg to 1000 mg.

The present disclosure further provides the use of the anti-CD40 antibody or antigen-binding fragment thereof, the multispecific antibody, the single-chain antibody or the pharmaceutical composition(s) comprising the same as described above, in the preparation of a medicament for the treatment of CD40-mediated or CD40L-mediated diseases or conditions; in particular the disease is cancer; in particular the cancer is selected from the group consisting of lymphoma, breast cancer, ovarian cancer, prostate cancer, pancreatic cancer, kidney cancer, lung cancer, liver cancer, gastric cancer, colorectal cancer, bladder cancer, rhabdomyosarcoma, esophageal cancer, cervical cancer, multiple myeloma, leukemia, gallbladder cancer, glioblastoma and melanoma.

The present disclosure further provides a method for treating and preventing CD40- or CD40L-mediated diseases or conditions, the method comprising contacting a subject with a prophylactically effective amount or a therapeutically effective amount of the anti-CD40 antibody or antigen-binding fragment thereof, the multispecific antibody, the single-chain antibody or the pharmaceutical composition(s) thereof as described above; in particular the disease or condition is cancer; in particular the cancer is selected from the group consisting of lymphoma, breast cancer, ovarian cancer, prostate cancer, pancreatic cancer, kidney cancer, lung cancer, liver cancer, gastric cancer, colorectal cancer, bladder cancer, rhabdomyosarcoma, esophageal cancer, cervical cancer, multiple myeloma, leukemia, gallbladder cancer, glioblastoma and melanoma.

The present disclosure further provides the use of the anti-CD40 antibody or antigen-binding fragment thereof, the multispecific antibody, the single-chain antibody or the pharmaceutical composition(s) thereof as described above in the preparation of a medicament for improving the symptom(s) of a patient suffering from autoimmune diseases.

The present disclosure further provides the use of the anti-CD40 antibody or antigen-binding fragment thereof, the multispecific antibody, the single-chain antibody or the pharmaceutical composition(s) thereof as described above in the preparation of a medicament for improving the symptom(s) of a patient suffering from inflammatory diseases.

### BRIEF DESCRIPTION OF THE DRAWINGS

FIG. 1 shows the activating effect of the murine anti-human CD40 antibodies on DC cells based on the CD80 activating molecule.

FIG. 2 shows the activating effect of the murine anti-human CD40 antibodies on DC cells based on the CD86 activating molecule.

FIG. 3 shows the tumor growth curve of Raji transplanted lymphoma, after co-transplanting Raji transplanted lymphoma with human PBMC and DC cells.

FIG. 4 shows the body weight change curve of NOG mice, after co-transplanting Raji transplanted lymphoma with human PBMC and DC cells into NOG mice.

FIG. 5 shows the tumor growth curve after intraperitoneal injection of a single dose of the anti-CD40 antibodies into mouse model of MC38 colon cancer.

FIG. 6 shows the activating effect of the anti-CD40 antibodies having mutation(s) in the heavy chain constant region on DC cells.

FIG. 7A and FIG. 7B show that the anti-CD40 antibodies having mutation(s) in the heavy chain constant region activate DC cells and promote cytokine production.

### DETAILED DESCRIPTION OF THE INVENTION

### 1. Terms

In order to more readily understand the present disclosure, certain technical and scientific terms are in particular defined below. Unless clearly indicated elsewhere in the document, all other technical and scientific terms used herein have the meaning commonly understood by one of ordinary skilled in the art to which the present disclosure pertains.

As used herein, the three-letter code and the single-letter code for amino acids are as described in J. Biol. Chem, 243, (1968) p 3558.

As used herein, “antibody” refers to immunoglobulin, a structure of four-peptide chains connected together by disulfide bonds between two identical heavy chains and two identical light chains. Different immunoglobulin heavy chain constant regions exhibit different amino acid compositions and sequence orders, hence present different kinds of antigenicity. Accordingly, immunoglobulins can be divided into five categories, or called as immunoglobulin isotypes, namely IgM, IgD, IgG, IgA and IgE; their corresponding heavy chains are μ chain, δ chain, γ chain, α chain and ε chain, respectively. According to its amino acid composition of hinge region and the number and location of heavy chain disulfide bonds, the same type of Ig can be divided into different sub-categories, for example, IgG can be divided into IgG1, IgG2, IgG3, and IgG4. Light chains can be divided into κ or λ chain, due to different constant regions. Each IgG among the five types has κ or λ chain.

In the present disclosure, the antibody light chain described herein further comprises a light chain constant region, which comprises a human or murine chain or variant(s) thereof.

In the present disclosure, the antibody heavy chain described herein further comprises a heavy chain constant region, which comprises human or murine IgG1, IgG2, IgG3, IgG4 or variant(s) thereof.

The sequence of about 110 amino acids close to the N-terminus of the antibody heavy and light chains, is highly variable, known as variable region (V region); the rest sequence of amino acid close to the C-terminus is relatively stable, known as constant region (C region). Variable region comprises three hypervariable regions (HVRs) and four relatively conserved framework regions (FRs). The three hypervariable regions determine the specificity of the antibody, also known as complementarity determining regions (CDRs). Each light chain variable region (VL) and each heavy chain variable region (VH) is composed of three CDRs and four FRs, with sequential order from the amino terminus to the carboxyl terminus being: FR1, CDR1, FR2, CDR2, FR3, CDR3, and FR4. The three light chain CDRs refer to LCDR1, LCDR2, and LCDR3; and the three heavy chain CDRs refer to HCDR1, HCDR2 and HCDR3.

The term “antigen-presenting cell” or “APC” is a cell which displays a foreign antigen to form a complex with MHC on its surface. T cells recognize this complex using the T cell receptor (TCR). Examples of APCs include, but are not limited to, dendritic cells (DC), peripheral blood mononuclear cells (PBMC), monocytes, B lymphoblasts and monocyte-derived dendritic cells (DC). The term “antigen presentation” refers to the process during which APCs capture antigens and make them to be recognized by T cells, for example as a component of MHC-I/MHC-II conjugates.

The term “CD40” includes any variant or isoform of CD40 that is naturally expressed by a cell. The antibodies of the present disclosure can be cross-reactive with CD40 from non-human species. Alternatively, the antibodies may also be specific for human CD40 and may not exhibit cross-reactivity with other species. CD40 or any variant or isoform thereof can be isolated from cells or tissues in which they are naturally expressed, or produced by recombinant techniques using common techniques in the art and those described herein. Preferably, the anti-CD40 antibodies target human CD40 having normal glycosylation pattern.

The term “murine antibody” in the present disclosure refers to a monoclonal antibody against human CD40 prepared according to the knowledge and skills in the art. During the preparation, the test subject is injected with CD40 antigen, and then the hybridoma expressing the antibody showing desired sequences or functional features is isolated. In a preferred embodiment of the present disclosure, the murine CD40 antibody or antigen-binding fragment thereof may further comprise light chain constant region of murine κ, λ chain or variant thereof, or further comprise heavy chain constant region of murine IgG1, IgG2, IgG3 or IgG4 or variant thereof.

The term “human antibody” includes antibodies having variable and constant regions of human germline immunoglobulin sequences. Human antibodies of the present disclosure can include amino acid residues that are not encoded by human germline immunoglobulin sequences (such as mutations introduced by random or site-specific mutagenesis in vitro or by somatic mutation in vivo). However, the term “human antibody” does not include such antibodies in which CDR sequences derived from another mammalian species germline, such as a mouse, have been grafted into human framework sequence (i.e. “humanized antibody”).

The term “humanized antibody”, also known as CDR-grafted antibody, refers to an antibody generated by grafting non-human CDR sequences into a variable region framework of a human antibody. Humanized antibody overcomes the strong immune response induced by the chimeric antibody that carries a large amount of heterologous protein components. To avoid the decrease in activity caused by reducing the immunogenicity, the variable region of the antibody is subjected to minimum back-mutation to maintain the activity.

The term “chimeric antibody”, is an antibody which is formed by fusing the variable region of a first species (such as murine) antibody with the constant region of another species (such as human) antibody, so as to alleviate the heterologous antibody-induced immune response. To establish a murine-human chimeric antibody, a hybridoma secreting specific murine monoclonal antibody is first established, variable region genes are then cloned from murine hybridoma cells, and then constant region genes of human antibody are cloned, the murine variable region genes are ligated with human constant region genes to form a chimeric gene which can be inserted into a human vector, and finally the chimeric antibody molecule is expressed in a eukaryotic or prokaryotic industrial system. The constant region of human antibody is selected from the heavy chain constant region derived from human IgG1, IgG2, IgG3 or IgG4 or variant(s) thereof; and preferably comprises heavy chain constant region derived from human IgG1 or IgG2.

The term “antigen-binding fragment”, referred to as antigen-binding fragments of an antibody or antibody analogs, usually comprises at least part of the antigen-binding regions or variable regions (for example, one or more CDRs) of a parental antibody. Antibody fragments retain at least partial binding specificity of the parent antibody. Generally, when the activity is expressed in mole, the antibody fragment retains at least 10% of the parent binding activity. Preferably, the antibody fragment retains at least 20%, 50%, 70%, 80%, 90%, 95% or more binding affinity of the parent antibody to the target. Examples of antigen-binding fragments include, but are not limited to: Fab, Fab′, F(ab′)₂, Fv fragment, linear antibody, single-chain antibody, nanobody, domain antibody, and multispecific antibody. Engineered variants of antibody are reviewed in Holliger and Hudson (2005) Nat. Biotechnol. 23: 1126-1136.

The “Fab fragment” consists of one light chain and one CH1 and variable region of heavy chain. The heavy chain of a Fab molecule cannot form a disulfide bond with another heavy chain molecule.

The “Fc” region comprises two heavy chain fragments having CH2 and CH3 domains of the antibody. The two heavy chain fragments are held together by two or more disulfide bonds and also by hydrophobic interaction of CH3 domain.

The “F(ab′)₂ fragment” comprises two light chains and two heavy chains comprising the portion of constant region between CH1 and CH2 domains, thereby forming an inter-chain disulfide bond between the two heavy chains. Therefore, F(ab′)2 fragment is composed of two Fab′ fragments held together by disulfide bond between the two heavy chains.

The “Fv region” comprises variable regions from both heavy and light chains, but lacks the constant region.

The term “multispecific antibody” is used in its broadest sense to encompass antibodies having multi-epitope specificity. These multispecific antibodies involve, but are not limited to, antibodies comprising a heavy chain variable region (VH) and a light chain variable region (VL), wherein the VH-VL unit has multi-epitope specificity; antibodies having two or more VL and VH regions, each VH-VL unit binding to different target or different epitope of the same target; antibodies having two or more single variable regions, each single variable region binding to different target or different epitope of the same target; full length antibodies, antibody fragments, diabodies, bispecific diabodies and triabodies, antibody fragments that have been covalently or non-covalently linked, and the like.

In the context of this application, when referring to a mutation position of the heavy chain constant region, the term “position(s) n corresponding to SEQ ID NO: m” or “position(s) n of SEQ ID NO: m” means: in different antibody numbering systems, a mutation site is comparable or equivalent to position n of SEQ ID NO: m, in terms of position. The skilled persons know that current antibody numbering systems include but are not limited to EU, Kabat, Chothia, IMGT (Lefranc, 2003) and AHo (Honegger and Plückthun, 2001) and so on. When a certain position is defined as position “n” according to one numbering system, it may be defined as position n′ according to another numbering system. The skilled persons can easily determine the corresponding relationship between specific sites according to different numbering systems (for example, EU numbering) based on common knowledge.

The term “antibody-drug conjugate” (ADC) refers to one or more chemically synthesized molecules (including but not limited to cytotoxic agents) conjugated to an antibody or an antibody fragment.

The term “single-chain antibody” is a single-chain recombinant protein linked by a linker peptide between the heavy chain variable region (VH) and the light chain variable region (VL) of an antibody. It is the smallest antibody fragment with complete antigen binding sites.

The term “domain antibody fragment” is an immunoglobulin fragment with immunological function, which only comprises a heavy chain variable region or a light chain variable region chain. In some cases, two or more VH regions are covalently linked to a peptide linker to form a bivalent domain antibody fragment. Two VH regions of the bivalent domain antibody fragment can target the same or different antigens.

The term “binding to CD40” in the present disclosure refers to the ability to interact with human CD40. The term “antigen-binding site(s)” in the present disclosure refers to a discrete three-dimensional spatial site on an antigen that can be recognized by the antibody or the antigen-binding fragment of present disclosure.

The term “epitope” refers to a site on an antigen that is specifically bound by an immunoglobulin or antibody. Epitopes may be formed from adjacent amino acids or nonadjacent amino acids but juxtaposed by tertiary folding of protein. Epitopes formed from adjacent amino acids are typically retained after exposure to denaturing solvent; however epitopes formed via tertiary folding are typically lost after treatment with denaturing solvent. Epitopes usually have a unique spatial conformation, including at least 3 to 15 amino acids. Methods for determining which epitope is bound by a given antibody are well known in the art, including immunoblotting and immunoprecipitation assays and the like. Methods for determining the spatial conformation of an epitope include techniques in the art and techniques described herein, such as X-ray crystallography and two-dimensional nuclear magnetic resonance and the like.

As used in the present disclosure, the terms “specifically bind” and “selectively bind” refer to the binding of an antibody to an epitope on a predetermined antigen. Typically, when human CD40 is used as an analyte and an antibody is used as a ligand, the antibody binds to a predetermined antigen at an equilibrium dissociation constant (K_{D}) of less than about 10⁻⁷ M or even less, as measured by surface plasmon resonance (SPR) techniques in an instrument, and the affinity of the antibody for binding to a predetermined antigen is at least twice higher than that for binding to a non-specific antigen other than the predetermined antigen or closely related antigen (such as BSA, etc). The term “antibody recognizing an antigen” can be used interchangeably herein with the term “antibody specifically binding to”.

The term “cross-reactivity” refers to the ability of an antibody of the present disclosure to bind to CD40 from a different species. For example, an antibody of the present disclosure that binds to human CD40 can also bind to CD40 from another species. Cross-reactivity is measured by detecting specific reactivity with purified antigens in binding assays (e.g., SPR and ELISA), or by detecting the binding or functional interactions with cells that express CD40 physiologically. Methods for determining cross-reactivity include standard binding assays as described herein, such as surface plasmon resonance (SPR) analysis, or flow cytometry.

The term “inhibiting” or “blocking” can be used interchangeably and encompasses both partial and complete inhibition/blocking. Preferably, the inhibition/blocking of a ligand can reduce the normal level or alter the type of activity when ligand binding occurs without inhibition or blocking. Inhibition and blocking are also intended to include any measurable decrease of ligand-binding affinity when contacted with an anti-CD40 antibody, compared to that when not contacted with an anti-CD40 antibody.

The term “inhibiting growth” (e.g., when referring to cells) is intended to include any measurable decrease in cell growth.

The terms “inducing an immune response” and “enhancing an immune response” are used interchangeably and refer to the stimulation (i.e., passive or adaptive) of an immune response to a particular antigen. In the context of CDC or ADCC, the term “induction” refers to the stimulation of particular direct cytotoxic mechanism.

As used in present disclosure, the term “ADCC”, namely antibody-dependent cell-mediated cytotoxicity, refers to that the cells expressing Fc receptors directly kill the target cells coated by an antibody by recognizing the Fc segment of the antibody. ADCC effector function of the antibody can be reduced or eliminated by modifying the Fc segment of IgG. The modification refers to mutations in antibody heavy chain constant region, such as mutations selected from N297A, L234A, L235A in IgG1; IgG2/4 chimera; or F235E or L234A/E235A mutation in IgG4.

Methods for producing and purifying antibodies and antigen-binding fragments are well known in the art and can be found, for example, in Antibody Experimental Technology Guide of Cold Spring Harbor, Chapters 5-8 and 15. For example, mice can be immunized with human CD40, or fragments thereof and the resulting antibodies can then be re-natured, purified and sequenced by using conventional methods well known in the art. Antigen-binding fragments can also be prepared by conventional methods. The antibody or the antigen-binding fragment of the present disclosure is genetically engineered to introduce one or more human framework regions (FRs) to a non-human derived CDR. Human FR germline sequences can be obtained from ImMunoGeneTics (IMGT) via their website http://imgt.cines.fr, or from The Immunoglobulin FactsBook, 20011SBN012441351.

The engineered antibody or antigen-binding fragment of the present disclosure may be prepared and purified using conventional methods. For example, cDNA sequence encoding the corresponding antibody may be cloned and recombined into a GS expression vector. The recombinant immunoglobulin expression vector may then stably transfect CHO cells. As a more recommended method well known in the art, mammalian expression system will result in glycosylation of antibody, typically at the highly conserved N-terminus in the FC region. Stable clones are obtained through expression of an antibody specifically binding to human antigen. Positive clones may be expanded in a serum-free culture medium for antibody production in bioreactors. Culture medium, into which an antibody has been secreted, may be purified and collected by conventional techniques. The antibody may be filtered and concentrated using common techniques. Soluble mixture and aggregate may be effectively removed by common techniques, including size exclusion or ion exchange. The obtained product may be immediately frozen, for example at −70° C., or may be lyophilized.

The antibody of the present disclosure refers to a monoclonal antibody. The monoclonal antibody (mAb) of the present disclosure refers to an antibody obtained from a single clone of cell strain, and the cell strain is not limited to a eukaryotic, a prokaryotic or a phage clonal cell strain. Monoclonal antibodies or antigen-binding fragments can be obtained recombinantly using, for example, hybridoma techniques, recombinant technique, phage display technique, synthetic technique (e.g., CDR-grafting), or other techniques in the prior art.

“Administration”, “administering” and “treatment,” as applied to an animal, human, experimental subject, cell, tissue, organ, or biological fluid, refer to contacting an exogenous pharmaceutical, therapeutic agent, diagnostic agent, or composition with the animal, human, subject, cell, tissue, organ, or biological fluid. “Administration”, “administering” and “treatment” can refer, e.g., to therapeutic, pharmacokinetic, diagnostic, research, and experimental methods. Treatment of a cell encompasses contacting a reagent with the cell, as well as contacting a reagent with a fluid, wherein the fluid is in contact with the cell. “Administration”, “administering” and “treatment” also means in vitro and ex vivo treatments, e.g., of a cell, by a reagent, diagnostic, composition, or by another cell. “Treatment” as it applies to a human, veterinary, or a subject to be studied, refers to therapeutic treatment, prophylactic or preventative measures, research and diagnostic applications.

“Treat” means to administer a therapeutic agent, such as a composition comprising any of the antibodies or antigen-binding fragment thereof in the present disclosure, internally or externally to a subject having one or more disease symptoms for which the agent has known therapeutic activity. Typically, the agent is administered in an amount effective to alleviate one or more disease symptoms in the treated subject or cohort of subjects, regardless of by inducing the regression of such symptom(s) or by inhibiting the progression to any clinically un-measurable degree.

The amount of a therapeutic agent that is effective to alleviate any particular disease symptom (also referred to “therapeutically effective amount”) may vary according to factors such as the disease state, age, and weight of the subject, and the ability of the agent to elicit a desired response in the subject. Whether a disease symptom has been alleviated can be assessed by any clinical measurement typically used by physicians or other skilled healthcare providers to assess the severity or progression status of that symptom. Even if an embodiment of the present disclosure (e.g., a treatment method or article of manufacture) is not effective in alleviating the disease symptom(s) of interest in every subject, it does alleviate the target disease symptom(s) of interest in a statistically significant number of subjects, as determined by any statistical test known in the art (such as, the Student's t-test, the chi-square test, the U-test according to Mann and Whitney, the Kruskal-Wallis test (H-test), Jonckheere-Terpstra-test and the Wilcoxon-test).

“Conservative modification” or “conservative substitution or replacement” means that an amino acid with similar characteristics (such as charge, side chain size, hydrophobicity/hydrophilicity, main chain conformation and rigidity, etc.) can be used to replace an amino acid in a protein; such substitution can be frequently performed without changing the biological activity of the protein. Those skilled in the art know that, generally speaking, substitution for a single amino acid in a non-essential region of a polypeptide does not substantially change the biological activity (see, for example, Watson et al. (1987) Molecular Biology of the Gene, The Benjamin/Cummings Pub. Co., page 224, 4^{th} edition). In addition, the substitution for amino acid having similar structure or function is unlikely to disrupt biological activity. The common conservative substitutions of amino acids are as follows:

| | | Preferred |
|---|---|---|
| Original residue | Exemplary substituents | substituents |
| Ala (A) | Val, Leu, Ile | Val |
| Arg (R) | Lys, Gln, Asn | Lys |
| Asn (N) | Gln, His, Asp, Lys, Arg | Gln |
| Asp (D) | Glu, Asn | Glu |
| Cys (C) | Ser, Ala | Ser |
| Gln (Q) | Asn, Glu | Asn |
| Glu (E) | Asp, Gln | Asp |
| Gly (G) | Ala | Ala |
| His (H) | Arg, Asn, Gln, Lys | Arg |
| Ile (I) | Leu, Val, Met, Ala, Phe, Norleucine | Leu |
| Leu (L) | Ile, Norleucine, Val, Met, Ala, Phe | Ile |
| Lys (K) | Arg, Gln, Asn | Arg |
| Met (M) | Leu, Phe, Ile | Leu |
| Phe (F) | Tyr, Leu, Val, Ile, Ala | Tyr |
| Pro (P) | Ala | Ala |
| Ser (S) | Thr | Thr |
| Thr (T) | Ser | Ser |
| Trp (W) | Tyr, Phe | Tyr |
| Tyr (Y) | Phe, Trp, Thr, Ser | Phe |
| Val (V) | Leu, Ile, Met, Phe, Ala, Norleucine | Leu. |

“Effective amount” involves an amount sufficient to ameliorate or prevent a symptom or sign of a medical condition. Effective amount also means an amount sufficient to allow or to facilitate diagnosis. An effective amount for a particular subject or veterinary subject may vary depending on factors such as the condition being treated, the general health of the subject, the route and dose of administration and the severity of side effects. An effective amount can be the maximal dose or dosing regimen that avoids significant side effects or toxic effects.

“Exogenous” refers to substances that are produced outside an organism, cell, or human body, depending on the background.

“Endogenous” refers to substances that are produced inside an organism, cell, or human body, depending on the background.

“Homology” refers to the sequence similarity between two polynucleotide sequences or between two polypeptides. When the positions in the two compared sequences are occupied by the same base or amino acid residue (for example, if each position of two DNA molecules is occupied by adenine), then the molecules are deemed to be homologous at that position. The percent homology between two sequences is a function of the number of matching or homologous positions shared by two sequences divided by the number of all positions to be compared×100%. For example, in an optimal sequence alignment, if 6 of the 10 positions in two sequences match with each other or are homologous, then the two sequences will be deemed as 60% homologous. Generally speaking, the comparison is performed, when two sequences are aligned to obtain the optimal percentage of homology.

As used herein, the expressions “cell,” “cell line,” and “cell culture” are used interchangeably and all such designations include its progeny. Thus, the term “transformed cell” refers to the primary subject cell and cultures derived therefrom without considering the number of passages. It is also understood that all progeny may not be precisely identical in the aspect of DNA component and/or content, due to deliberate or indeliberate mutations. Mutant progeny that have the same function or biological activity as that of original cell are also covered by this term.

“Optional” or “optionally” means that the event or situation that follows may but not necessarily occur, and the description includes the instances in which the event or circumstance occurs or does not occur. For example, “optionally comprises 1 to 3 antibody heavy chain variable region(s)” means the antibody heavy chain variable region with specific sequence can be, but not necessarily, present.

### EXAMPLES

The following examples are used to further describe the present invention, but these examples do not limit the scope of the present invention. The experimental methods that do not specify specific conditions in the examples of the present invention usually follow conventional conditions, such as Antibodies: A Laboratory Manual, Molecular Cloning Manual from Cold Spring Harbor; or according to the conditions recommended by the manufacturer of materials or products. The reagents for which the sources are not specifically indicated are conventional reagents commercially available.

### Example 1 the Sequences and Preparation of Immune-Antigen and Screening-Antigen

His-tagged human CD40 (h-CD40-his) recombinant protein, Fc-tagged human CD40 (h-CD40-Fc) recombinant protein, His-tagged mouse CD40 (m-CD40-his) recombinant protein and His-tagged rhesus monkey CD40 (rhesus-CD40-his) recombinant protein (#CD0-052H7) were all purified commercial protein reagents purchased from Acrobiosystems, and the each sequence source is shown in Table 1. The protein reagents can be used in each test of the following examples.

TABLE 1
Sources for amino acid sequences of recombinant proteins

| | Amino acid sequence (from | |
|---|---|---|
| Name | the beginning to the end) | Genbank accession No. |
| h-CD40-his | Glu21-Arg193 | AAH12419.1 |
| h-CD40-Fc | Glu21-Arg193 | NP_001241.1 |
| m-CD40-his | Val24-Arg193 | P27512 |
| rhesus-CD40-his | Glu21-Arg193 | NP_001252791.1 |

### Example 2 Preparation of Antibody Hybridoma

Anti-human CD40 monoclonal antibody was produced by immunizing mice. Laboratory C57BL/6 mouse: female, 6 to 8 week-old (JOINN Laboratories (Suzhou) New Medicament Research Center Co., Ltd., animal production license number: 201503259). Breeding environment: SPF level.

After the mice being purchased, they were kept in a laboratory environment for 1 week, adjusted to 12/12 hours light/dark cycle; at a temperature of 20-25° C.; with humidity of 40-60%. The adapted mice were assigned into 2 cages, 5 in each cage.

The immune-antigen is a modified human-CD40 recombinant protein with an Fc tag (h-CD40-Fc, prepared in a phosphate buffer solution, at 1 μg/μl). Emulsification was performed with Freund's adjuvant (Sigma, Lot No.: F5881/F5506): Freund's complete adjuvant (CFA) for the first emulsification; and nucleic acid adjuvant (CpG, Sangon Biotech) and injectable aluminum (Imject Alum, Thermo, Lot No.: PH203866) for the rest booster immunizations. The immunization date was on day 0, 14, 28, 42, 56, and 70. Blood was collected for blood test on day 21, 35, 49, 63, and 77. The mouse serum was detected by ELISA method to determine the antibody titer in the mouse serum.

After the fourth immunization, mouse with a high and stable antibody titer was selected for spleen cell fusion. 3 days before fusion, booster immunization was performed by intraperitoneal (IP) injection of 10 μg/mouse of antigen formulated in phosphate buffer solution. Optimized PEG-mediated fusion steps were used to fuse splenic lymphocytes and myeloma cells Sp2/0 cells (ATCC® CRL-8287™) to obtain hybridoma cells, and five monoclonal hybridoma cell lines showing favorable in vitro activity were selected.

### Example 3 ELISA Binding Assay

ELISA assay was used to detect the binding properties of anti-CD40 antibodies. CD40 recombinant protein was directly coated with his tag. After the antibody was added, a secondary antibody (HRP-conjugated anti-Fc antibody) and HRP substrate TMB were added to detect the binding activity of the antibody to the antigen.

Human or rhesus monkey CD40-his protein was coated onto a 96-well micro-titer plate, 100 μl per well at a concentration of 0.5 μg/mL, and incubated overnight at 4° C. The plate was washed with washing buffer for three times, 250 μl per well. The plate was shaken for 10 seconds during each washing to ensure sufficient washing. 200 μl/well blocking solution was added and incubated at room temperature for 2 hours. The plate was washed with washing buffer for three times, 250 μl per well. The plate was shaken for 10 seconds during each washing to ensure sufficient washing. 100 μl of anti-CD40 antibody to be tested diluted with diluent was added into each well and incubated for 1 hour at room temperature. The plate was washed with washing buffer for three times, 250 μl per well. 100 μl of HRP-labeled goat anti-human IgG secondary antibody diluted at 1:20000 with a diluent was added to each well, and incubated for 1 hour at room temperature. The plate was washed with washing buffer for three times, 250 μl per well. 100 μl TMB was added to each well, and reaction was performed for 15 minutes in the dark. 50 μl of 0.16M sulfuric acid was added into each well. Thermo MultiSkanFc microplate reader was used to read OD value under 450 nm, and EC50 value for each CD40 antibody to binding to CD40 was calculated.

TABLE 2
Results of ELISA binding assay of murine hybridoma
antibodies against CD40 from different germ lines

| | ELISA EC50 (ng/mL) | | |
|---|---|---|---|
| Antibody strain | hCD40-his | Rhesus CD40-his | murine CD40-his |
| 1D9 | 10.01 | 9.808 | no binding |
| 2H6 | 7.063 | 7.207 | no binding |
| 9E5 | 5.996 | 6.704 | no binding |
| 14C10 | 8.808 | 9.494 | no binding |
| 38B4 | 12.9 | 11.81 | no binding |

### Example 4 Test of Anti-Cd40 Antibody Blocking the Binding Between Cd40 and Cd40L

In this test, through an in vitro blocking assay, the anti-human CD40 antibodies thus screened were tested for their blocking the binding between human CD40 and human CD40L.

The particular method was as follows: the Fc-tagged CD40 recombinant protein (h-CD40-Fc) was coated onto a 96-well micro-titer plate, anti-CD40 antibody was added to fully bind to and occupy the epitopes, and then his-tagged CD40L was added, and His tag was detected to calculate the amount of CD40 binding to CD40L, and the IC50 value for CD40 antibody to block the CD40 activity sites was calculated.

Human CD40-Fc protein was coated onto a 96-well micro-titer plate, 100 μl per well at a concentration of 1 μg/mL, and incubated overnight at 4° C. The plate was washed with washing buffer for three times, 250 μl per well. The plate was shaken for 10 seconds during each washing to ensure sufficient washing. 200 μl/well blocking solution was added and incubated at room temperature for 2 hours. The plate was washed with washing buffer for three times, 250 μl per well. The plate was shaken for 10 seconds during each washing to ensure sufficient washing. 100 μl of anti-CD40 antibody to be tested diluted with diluent was added into each well and incubated for 1 hour at room temperature. The plate was washed with washing buffer for three times, 250 μl per well. 100 μl of diluted CD40L-his was added into each well, and incubated for 1 hour at room temperature. The plate was washed with washing buffer for three times. 100 μl of HRP-labeled anti-his tag secondary antibody diluted at 1:2000 with a diluent was added to each well, and incubated for 1 hour at room temperature. The plate was washed with washing buffer for three times, 250 μl per well. 100 μl TMB was added to each well, and reaction was performed for 15 minutes in the dark. 50 μl of 0.16M sulfuric acid was added into each well. Thermo MultiSkanFc microplate reader was used to read OD value under 450 nm, and IC50 value for CD40 antibody to block the binding of CD40 to CD40L was calculated.

TABLE 3
Results of ELISA assay for blocking
the binding of human hCD40/hCD40L

| Antibody strain | IC50 (g/mL) |
|---|---|
| 1D9 | 0.2634 |
| 2H6 | 0.2682 |
| 9E5 | 0.2787 |
| 14C10 | 0.3001 |
| 38B4 | 0.2934 |

### Example 5 Determination of Affinity by Biacore

According to the method described in the instruction available from Human Anti-capture Kit (Cat.# BR-1008-39, GE), human anti-capture antibody was covalently coupled to biosensing chip CM5 of the Biacore instrument (Biacore X100, GE), a certain amount of chimeric or humanized antibodies to be tested was affinity-captured, and then a series of concentration gradients of CD40 antigen (CD40 antigen purchased from Acrobiosystems) flowed through the surface of the chip. Biacore instrument (Biacore X100, GE) was used to detect the reaction signal in real-time, thus to obtain the association and dissociation curves. After each cycle of dissociation was completed, the biochip was washed and regenerated with a regeneration solution provided by the Human Anti-capture Kit. The amino coupling kit used in the test was purchased from GE (Cat. # BR-1000-50, GE), and HBS-EP+10× buffer solution (Cat. # BR-1006-69, GE) was diluted to 1×(pH 7.4) with double distilled water.

The data obtained from the test was fitted against a (1:1) Binding model using BiacoreX100 evaluation software2.0 GE, and the affinity value was obtained, as shown in Table 10 and Table 11.

### Example 6 Test of Activity of Anti-Cd40 Antibody on Reporter Gene in Cells

HEK-Blue CD40L cells were purchased from Invivogen (Cat#hkb-cd40). The cells were stably transfected with human CD40 gene and NF-kB-mediated SEAP genome. SEAP secreted in the supernatant can be detected by SEAP substrate QUANTI-Blue, to characterize the activation level of CD40 signaling pathway. In this test, the activation of HEK-Blue CD40L cells was detected, and the in vitro activity of CD40 antibodies was evaluated in cell according to EC50.

The HEK-Blue CD40L cells were cultivated in DMEM medium comprising 10% FBS, 100 μg/mL Zeocin and 30 μg/mL Blasticidin, and sub-cultured for 2 to 3 times per week at a passage ratio of 1:5 or 1:10. During sub-culturing, the medium was removed, the cell layer was rinsed with 5 mL of 0.25% trypsin, then the trypsin was removed, the cells were incubated in an incubator for 3 to 5 minutes, and then fresh medium was added to re-suspend the cells. 100 μL of cell suspension was added to a 96-well cell culture plate at a density of 5×10⁵ cells/mL. The medium was DMEM comprising 10% FBS, 100m/mL bleomycin Zeocin and 30 μg/mL blasticidin, and 100 μl of sterile water alone was added to the periphery wells of the 96-well plate. The culture plate was incubated in an incubator for 24 hours (37° C., 5% CO₂). Once the adherence of cells was observed, 100 μl of the antibody to be tested at a gradient of dilutions was added to each well. The culture plate was incubated in an incubator for 20-24 hours (37° C., 5% CO₂). 40 μl of cell supernatant was transferred from each well to anew 96-well flat bottom plate, 160 μl QUANTI-Blue substrate solution was added, and the culture plate was incubated in an incubator in the dark for 1-3 hours. The absorbance at 620 nm was measured with a microplate reader (Thermo MultiSkanFc), and EC50 value was calculated to evaluate the activity of the CD40 antibody in vitro in cells.

TABLE 4
Test results of activity of anti-CD40
antibody on reporter gene in cells

| Antibody strain | test of activity in HEK293-CD40L cells, EC50 (g/mL) | |
|---|---|---|
| 1D9 | +++ | 0.01454 |
| 2H6 | +++ | 0.01511 |
| 9E5 | ++ | 0.01712 |
| 14C10 | +++ | 0.01087 |
| 38B4 | ++ | 0.0365 |

### Example 7 Test of Anti-Cd40 Antibody to Activate Dc Cells

PBMCs were isolated from the peripheral blood of normal human subject, and then monocytes were sorted using CD14 MACS beads. RPMI 1640 medium comprising 10 ng/mL IL4 and 100 ng/mL GM-CSF was added for cultivation for 6 days to induce MoDC cells (dendritic cells derived from monocytes). Cells were collected after 6 days, 1×10⁵ cells were taken, and stained with CD209-PE, CD1a-PerCP/Cy5.5 and CD14-PE/Cy7 for analyzing whether MoDC has been successfully induced by FACS (the above operations are routine operations in the art).

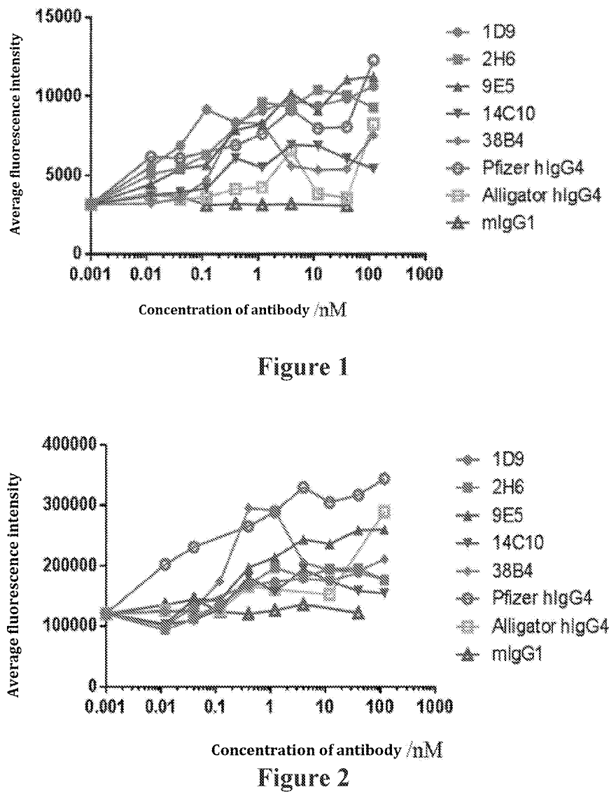

The successfully induced DCs were collected, each antibody to be tested and control antibody were added, and the corresponding dilution gradient of concentrations were set up (see FIG. 1 for the gradient of concentrations of antibody). After cultivating for 48 hours, the cells were collected and stained for CD80, CD86 and HLA-DR, and data was collected by FACS.

According to the data in a test of activating primary DC cells, all of the five murine antibodies showed obvious activity on activating molecules CD80 and CD86 on the surface of DC cells, in a dose-dependent way. The overall effect was comparable to, equivalent to, or even slightly better than that of the two control antibodies (CP-870,893 from Pfizer, and ADC-1013 from Alligator Bioscience) (See FIG. 1 and FIG. 2).

### Example 8 Cloning and Sequencing of Anti-Cd40 Antibody

The hybridoma subclones of the 5 antibodies identified from the above screening were taken, the hybridoma cells at logarithmic growth phase were collected; RNA was extracted with Trizol (Invitrogen, 15596-018) (following the instructions in the kit), and reverse transcription (PrimeScript™ Reverse Transcriptase, Takara, cat #2680A) was performed. The cDNA obtained by reverse transcription was amplified by PCR using mouse Ig-Primer Set (Novagen, TB326 Rev.B0503), and delivered to a company for sequencing. Finally, the sequences of 5 murine antibodies were obtained.

(1) The sequences of heavy chain and light chain variable region of murine monoclonal antibody 2H6 are as follows:

2H6 HCVR
(SEQ ID NO: 1)
QVQLQQSGAELVRPGTSVKVSCKASGYAFSDYLIEWAKQRPGQGLEWI
GVINPGSGGSNYNEKIKDRATLTADKSSSTAYMQLSSLTSEDSAVYFC
ARGGGGFTYVVGQGTLVTVSA;
2H6 LCVR
(SEQ ID NO: 2)
EIQLTQTTSSLSASLGDRVTISCRASQDISNYLNWYQQKPDGTIKLLL
NFASRLHSGVPSRFSGSGSGTDFFLTISNLEQDDIATYFCQQGSTLPW
TFGGGTKLEIK;

The CDR sequences included therein are shown in Table 5 below:

TABLE 5
CDR sequences of 2H6

| Name | Sequence | SEQ ID NO |
|---|---|---|
| HCDR1 | GYAFSDYLIE | SEQ ID NO: 3 |
| HCDR2 | VINPGSGGSNYNEKIKD | SEQ ID NO: 4 |
| HCDR3 | GGGGFTY | SEQ ID NO: 5 |
| LCDR1 | RASQDISNYLN | SEQ ID NO: 6 |
| LCDR2 | FASRLHS | SEQ ID NO: 7 |
| LCDR3 | QQGSTLPWT | SEQ ID NO: 8 |

(2) The sequences of heavy chain and light chain variable region of 9E5 are as follows:

9E5 HCVR
(SEQ ID NO: 9)
QVQLQQPGADLVKPGASVKMSCKASGYILTTYWITWVKQRPGQGLEWI
GDIHPGSGSTKYNEKFKSKATLTVDTSSSTAYMQLTRLSSEDSAVYYC
ARRDYWGQGTTLTVSS;
9E5 LCVR
(SEQ ID NO: 10)
DVLMTQSPLSLPVSLGDQASISCRSSQNIVNSQGNTYLEWYLQKPGES
PKLLIYKVTNRFSGVPDRFSGSGSGTDFTLKISRVEAEDLGVYYCFQA
SLVPWTFGGGTKLEIK;

The CDR sequences included therein are shown in Table 6 below:

TABLE 6
CDR sequences of 9E5

| Name | Sequence | SEQ ID NO |
|---|---|---|
| HCDR1 | GYILTTYWIT | SEQ ID NO: 11 |
| HCDR2 | DIHPGSGSTKYNEKFKS | SEQ ID NO: 12 |
| HCDR3 | RDY | SEQ ID NO: 13 |
| LCDR1 | RSSQNIVNSQGNTYLE | SEQ ID NO: 14 |
| LCDR2 | KVTNRFS | SEQ ID NO: 15 |
| LCDR3 | FQASLVPWT | SEQ ID NO: 16 |

(3) The sequences of heavy chain and light chain variable region of 1D9 are as follows:

1D9 HCVR
(SEQ ID NO: 37)
QVRLQQSGAELVRPGTSMRVSCKASGYAFTNYLINWVKQRPGQGLEWI
GILNPGSGGTNYNENFKDKATLTADKSSNTAYMQLSSLTSEDSAVYFC
IRGSPGFAYWGQGTLVTVSA;
1D9 LCVR
(SEQ ID NO: 38)
DIQMTQTTSSLSASLGDRVTISCRASQDINIYLNWYQQKPDGTVKLLI
YSTSGLHSGVPSRFNGSGSGTDYSLTISNLEQEDIATYFCQQGYTLPY
TFGGGTKLEIK;

The CDR sequences included therein are shown in Table 7 below:

TABLE 7
CDR sequences of 1D9

| Name | Sequence | SEQ ID NO |
|---|---|---|
| HCDR1 | GYAFTNYLIN | SEQ ID NO: 39 |
| HCDR2 | ILNPGSGGTNYNENFKD | SEQ ID NO: 40 |
| HCDR3 | GSPGFAY | SEQ ID NO: 41 |
| LCDR1 | RASQDINIYLN | SEQ ID NO: 42 |
| LCDR2 | STSGLHS | SEQ ID NO: 43 |
| LCDR3 | QQGYTLPYT | SEQ ID NO: 44 |

(4) The sequences of heavy chain and light chain variable region of 14C10 are as follows:

14C10 HCVR
(SEQ ID NO: 45)
QVQVQQSGAELVRPGTSVKVSCKASGYAFTNYLIEWVKQRPGQGLEWI
GVINPEFGGTNYNEKFKGKATLTADKSSSTAYMQLSSLTSEDSAVYFC
ARGGGGFTYWGQGTLVTVSA;
14C10 LCVR
(SEQ ID NO: 46)
HIQMTQTTSSLSASLGDRVTISCRASQDISSHLNWYQQKPDGTVKLLI
SYTSRLHSGVPSRFSGSGSGADYSLTISNLEQEDIATYFCQQGNTLPW
TFGGGTKLEIK;

The CDR sequences included therein are shown in Table 8 below:

TABLE 8
CDR sequences of 14C10

| Name | Sequence | SEQ ID NO |
|---|---|---|
| HCDR1 | GYAFTNYLIE | SEQ ID NO: 47 |
| HCDR2 | VINPEFGGTNYNEKFKG | SEQ ID NO: 48 |
| HCDR3 | GGGGFTY | SEQ ID NO: 49 |
| LCDR1 | RASQDISSHLN | SEQ ID NO: 50 |
| LCDR2 | YTSRLHS | SEQ ID NO: 51 |
| LCDR3 | QQGNTLPWT | SEQ ID NO: 52 |

(5) The sequences of heavy chain and light chain variable region of 38B4 are as follows:

38B4 HCVR
(SEQ ID NO: 53)
QVRLKQSGAELVRPGASVKVSCKASGYTFTDYYINWVKQRPGQGLEWI
AGIYPGTGNTYYNEKFKGKATLTAERSSSTAYMQLTSLTSEDSAVYFC
TRRGLPSLCFDYWGQGTTLTVSS;
38B4 LCVR
(SEQ ID NO: 54)
DFQMTQTTSSLSASLGDRVTISCSASQGISNYLNWYQQKPDGTVKLLI
YYTSSLHSGVPSRFSGSGSGTDYSLTISNLEPEDIATYYCQQYSKLPP
TFGGGTKLEIK;

The CDR sequences included therein are shown in Table 9 below:

TABLE 9
CDR sequences of 38B4

| Name | Sequence | SEQ ID NO |
|---|---|---|
| HCDR1 | GYTFTDYYIN | SEQ ID NO: 55 |
| HCDR2 | GIYPGTGNTYYNEKFKG | SEQ ID NO: 56 |
| HCDR3 | RGLPSLCFDY | SEQ ID NO: 57 |
| LCDR1 | SASQGISNYLN | SEQ ID NO: 58 |
| LCDR2 | YTSSLHS | SEQ ID NO: 59 |
| LCDR3 | QQYSKLPPT | SEQ ID NO: 60 |

Among them, the optimal two strains of antibodies (2H6 and 9E5) were selected for follow-up development. The obtained variable region sequences were respectively connected to human antibody IgG1 constant region sequences to obtain human-mouse chimeric antibody sequences. Using molecular cloning technology, the sequence of the chimeric antibody was inserted into pCP expression vector (purchased from Mabspace Biosciences), and then the sequence was identified by PCR (molecular cloning and other molecular biological operations in this part are carried out according to conventional operation conditions. For more details, please refer to “Molecular Cloning: A Laboratory Manual”). HEK293 cell expression system was used to obtain human-mouse chimeric antibodies 2H6-C and 9E5-C.

The chimeric antibodies purified by MabSelect SuRe affinity chromatography (GE Lifesciences) were tested for various activities in vitro. The data are shown in Table 10.

TABLE 10
In vitro activity of chimeric antibodies

| | | human | | |
|---|---|---|---|---|
| | | hCD40/ | | |
| | human | hCD40L, | | |
| | CD40-his | blocking | HEK293- | Biacore |
| | ELISA | ELISA | CD40 | affinity |
| | EC50 | IC50 | cell-binding | K_{D} |
| Chimeric antibody | (ng/mL) | (g/mL) | EC50 (g/mL) | (M) |
| 2H6-C | 4.565 | 0.6275 | 0.02593 | 3.98 |
| 9E5-C | 1.346 | 0.1218 | 0.03333 | 2.68 |
| Pfizer control | 5.628 | 0.2583 | 0.01638 | 20.35 |
| (hIgG4) | | | | |
| Alligator | 3.288 | 0.7233 | 0.39650 | 65.9 |
| control | | | | |
| (hIgG1) | | | | |

### Example 9 Humanization Test of Murine Antibody

Based on the obtained typical VH/VLCDR structures of the murine antibodies 2H6 and 9E5, the heavy chain variable region and light chain variable region sequences were aligned against an antibody germline database to obtain a human germline template with high homology.

The human germline light chain framework region is derived from the human kappa light chain gene. The human germline light chain template for the antibody of present disclosure is preferably Vk1-33/JK4 (for 2H6) or Vk2-28/JK4 (for 9E5).

The human germline heavy chain framework region is derived from the human heavy chain. The human germline heavy chain template for the antibody of present disclosure is preferably VH1-69/JH6 (for 2H6) or VH1-2/JH6 (for 9E5), as shown below:

Preferable human germline heavy chain template IGHV1-69 for 2H6

(SEQ ID NO: 21)
QVQLVQSGAEVKKPGSSVKVSCKASGGTFSSYAISWVRQAPGQGLEWM
GGIIPIFGTANYAQKFQGRVTITADKSTSTAYMELSSLRSEDTAVYYC
AR;

Preferable human germline light chain template IGkV1-33 for 2H6

(SEQ ID NO: 22)
DIQMTQSPSSLSASVGDRVTITCQASQDISNYLNWYQQKPGKAPKLLI
YDASNLETGVPSRFSGSGSGTDFTFTISSLQPEDIATYYCQQYDNLP;

Preferable human germline heavy chain template IGHV1-2 for 9E5

(SEQ ID NO: 23)
QVQLVQSGAEVKKPGASVKVSCKASGYTFTGYYMHWVRQAPGQGLEWM
GWINPNSGGTNYAQKFQGRVTMTRDTSISTAYMELSRLRSDDTAVYYC
AR;

Preferable human germline light chain template IGkV2-28 for 9E5

(SEQ ID NO: 24)
DIVMTQSPLSLPVTPGEPASISCRSSQSLLHSNGYNYLDWYLQKPGQS
PQLLIYLGSNRASGVPDRFSGSGSGTDFTLKISRVEAEDVGVYYCMQA
LQTP;

The CDR regions of the murine antibody were grafted onto the selected humanized template, to replace the humanized variable regions, and then recombined with corresponding human IgG constant regions (preferably IgG1 for heavy chain; and kappa for light chain). Based on the three-dimensional structure of the murine antibody, back-mutations were performed on the embedded residues, the residues that directly interact with CDR regions, and the residues that have an important impact on conformation of VL and VH, and the amino acid residues in CDR regions that are not chemically stable were optimized to obtain the final humanized molecules.

The sequences of the heavy chain variable regions are shown in SEQ ID NOs: 25-30;

The sequences of the light chain variable regions are shown in SEQ ID NOs: 31-36.

hu2H6-H1a:
(SEQ ID NO: 25)
QVQLVQSGAEVKKPGSSVKVSCKASGGTFSDYLIEWVRQAPGQGLEWM
GVINPGSGGSNYNEKIKDRVTITADKSTSTAYMELSSLRSEDTAVYYC
ARGGGGFTYWGQGTLVTVSS;
hu2H6-H1b:
(SEQ ID NO: 26)
QVQLVQSGAEVKKPGSSVKVSCKASGYAFSDYLIEWVRQAPGQGLEWM
GVINPGSGGSNYNEKIKDRVTLTADKSTSTAYMELSSLRSEDTAVYYC
ARGGGGFTYWGQGTLVTVSS;
hu2H6-H1c:
(SEQ ID NO: 27)
QVQLVQSGAEVKKPGSSVKVSCKASGYAFSDYLIEWVRQAPGQGLEWI
GVINPGSGGSNYNEKIKDRATLTADKSTSTAYMELSSLRSEDTAVYYC
ARGGGGFTYWGQGTLVTVSSFGQGTKLEIK;
hu9E5-H1a:
(SEQ ID NO: 28)
QVQLVQSGAEVKKPGASVKVSCKASGYTFTTYWITWVRQAPGQGLEWM
GDIHPGSGSTKYNEKFKSRVTMTVDTSISTAYMELSRLRSEDTAVYYC
ARRDYWGQGTTVTVSS;
hu9E5-H1b:
(SEQ ID NO: 29)
QVQLVQSGAEVKKPGASVKVSCKASGYTFTTYWITWVRQAPGQGLEWM
GDIHPGSGSTKYNEKFKSRVTLTVDTSISTAYMELSRLRSEDTAVYYC
ARRDYWGQGTTVTVSS;
hu9E5-H1c:
(SEQ ID NO: 30)
QVQLVQSGAEVKKPGASVKVSCKASGYILTTYWITWVRQAPGQGLEWM
GDIHPGSGSTKYNEKFKSRVTLTVDTSISTAYMELSRLRSEDTAVYYC
ARRDYWGQGTTVTVSS;
hu2H6-L1a:
(SEQ ID NO: 31)
DIQMTQSPSSLSASVGDRVTITCRASQDISNYLNWYQQKPGKAPKLLL
NFASRLHSGVPSRFSGSGSGTDFTFTISSLQPEDIATYYCQQGSTLPW
TFGGGTKVEIK;
hu2H6-L1b:
(SEQ ID NO: 32)
DIQLTQSPSSLSASVGDRVTITCRASQDISNYLNWYQQKPGKAPKLLL
NFASRLHSGVPSRFSGSGSGTDFTFTISSLQPEDIATYYCQQGSTLPW
TFGGGTKVEIK;
hu2H6-L1c:
(SEQ ID NO: 33)
DIQLTQSPSSLSASVGDRVTITCRASQDISNYLNWYQQKPGKTIKLLL
NFASRLHSGVPSRFSGSGSGTDFTFTISSLQPEDIATYYCQQGSTLPW
TFGGGTKVEIK;
hu9E5-L1a:
(SEQ ID NO: 34)
DIVMTQSPLSLPVTPGEPASISCRSSQNIVNSQGNTYLEWYLQKPGQS
PQLLIYKVTNRFSGVPDRFSGSGSGTDFTLKISRVEAEDVGVYYCFQA
SLVPWTFGGGTKVEIK;
hu9E5-L1b:
(SEQ ID NO: 35)
DVVMTQSPLSLPVTPGEPASISCRSSQNIVNSQGNTYLEWYLQKPGQS
PQLLIYKVTNRFSGVPDRFSGSGSGTDFTLKISRVEAEDVGVYYCFQA
SLVPWTFGGGTKVEIK;
hu9E5-L1c:
(SEQ ID NO: 36)
DVLMTQSPLSLPVTPGEPASISCRSSQNIVNSQGNTYLEWYLQKPGQS
PQLLIYKVTNRFSGVPDRFSGSGSGTDFTLKISRVEAEDVGVYYCFQA
SLVPWTFGGGTKVEIK.

By expression test of the above combinations of light and heavy chains and by comparisons between different numbers of back mutations, the humanized antibody molecules hu2H6 (with H1b heavy chain and L1c light chain) and hu9E5 (with H1c heavy chain and L1a light chain) were finally selected, and the respective complete light chain and heavy chain sequences are shown in SEQ ID NO: 17-20.

hu2H6 HC:
(SEQ ID NO: 17)
QVQLVQSGAEVKKPGSSVKVSCKASGYAFSDYLIEWVRQAPGQGLEWM
GVINPGSGGSNYNEKIKDRVTLTADKSTSTAYMELSSLRSEDTAVYYC
ARGGGGFTYWGQGTLVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPGK;
hu2H6 LC:
(SEQ ID NO: 18)
DIQLTQSPSSLSASVGDRVTITCRASQDISNYLNWYQQKPGKTIKLLL
NFASRLHSGVPSRFSGSGSGTDFTFTISSLQPEDIATYYCQQGSTLPW
TFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC;
hu9E5 HC:
(SEQ ID NO: 19)
QVQLVQSGAEVKKPGASVKVSCKASGYILTTYWITWVRQAPGQGLEWM
GDIHPGSGSTKYNEKFKSRVTLTVDTSISTAYMELSRLRSEDTAVYYC
ARRDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKG
QPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYT
QKSLSLSPGK;
hu9E5 LC:
(SEQ ID NO: 20)
DIVMTQSPLSLPVTPGEPASISCRSSQNIVNSQGNTYLEWYLQKPGQS
PQLLIYKVTNRFSGVPDRFSGSGSGTDFTLKISRVEAEDVGVYYCFQA
SLVPWTFGGGTKVEIKRTVAAPSVFIFPPSDEQLKSGTASVVCLLNNF
YPREAKVQWKVDNALQSGNSQESVTEQDSKDSTYSLSSTLTLSKADYE
KHKVYACEVTHQGLSSPVTKSFNRGEC.

### Example 10 Test Data of Humanized Antibody

The present disclosure shows the binding activity and blocking activity of humanized antibodies hu2H6 and hu9E5 to human CD40 and rhesus CD40, as shown in Table 11.

The results show that the humanized anti-human CD40 antibodies of present disclosure have ELISA binding and blocking activity comparable to that of positive antibodies Pfizer/Alligator. In particular, the affinity of hu9E5 to human CD40 measured by Biacore is 10 times or more than that of the antibody Alligator, a positive control, and 4 times or more than that of the Pfizer Control.

TABLE 11
In vitro activities of humanized antibodies hu2H6 and hu9E5

| | | | human | | |
|---|---|---|---|---|---|
| | | | hCD40/ | | |
| | human | Rhesus | hCD40L, | | |
| | CD40-his | CD40-his | blocking | HEK293- | Biacore |
| | ELISA | ELISA | ELISA | CD40 | affinity |
| | EC50 | EC50 | IC50 | cell-binding | K_{D} |
| | (ng/mL) | (ng/mL) | (g/mL) | EC50 (g/mL) | (M) |
| Hu2H6-11 | 3.680 | 2.945 | 0.6735 | 0.01538 | 1.120E−8 |
| Hu9E5-25 | 1.650 | 1.661 | 0.3084 | 0.13970 | 5.301E−9 |
| Alligator | 1.293 | 1.243 | 0.6471 | 1.36200 | 1.66E−7 |
| control | | | | | |
| (hIgG1) | | | | | |
| Pfizer | 3.976 | 3.561 | 0.3106 | 0.01907 | 2.035E−8 |
| control | | | | | |
| (hIgG4) | | | | | |

### Example 11 Inhibition of Mouse Tumor Growth by Anti-Cd40 Antibody

Peripheral blood of normal human subject was taken, and PBMCs of healthy human subject were separated by density gradient centrifugation. The monocytes were isolated with CD14+ microbeads kit, the CD14+ monocytes were isolated according to procedure provided by the kit, i.e., 20 μl anti-CD14 microbeads were added to every 10⁷ cells, and incubated at 4° C. for 15 minutes. Then, the cells were added to magnetic column, and the column was washed for three times, the cells were collected from the magnetic column, namely CD14+ monocytes. CD14+ monocytes were added with RPMI 1640 medium comprising 10 ng/mL IL-4 and 100 ng/mL GM-CSF, and were cultivated for 6 days (the method for cultivating is a conventional method in the art); then the MoDC cells were induced and cultivated, and the remaining cells were added with RPMI 1640 comprising IL-2; the suspended cells were collected after cultivating (the method for cultivating and the method for collecting cells are conventional methods in the art), T cells were sorted by CD3+ microbead kit. Six days later, MoDC cells and CD3+ T cells were collected and separated; and mixed with Raji cells (Cell Bank of Shanghai Academy of Biological Sciences, cultivated in RPMI1640 medium comprising 10% fetal bovine serum) at a ratio of 1:5:20. The mixture was used to subcutaneously inoculate each NOG mouse (Nanjing Galaxy Biopharma Co., Ltd, adaptive breeding for 5 days). The laboratory animals were kept in an independent ventilated cage with constant temperature and humidity. The temperature in the breeding room was 18.0-26.0° C., the humidity was 40-70%, and the ventilation rate was 10-20 times per hour. The alternating time for day and night was 12h/12h.

Human IgG1 antibody control group, hu2H6, hu9E5 and control antibody G12 group (i.e. ADC-1013 from Alligator Bioscience) were divided in the test, and the dose was 3 mg/kg for each group. Each group of 5 mice was injected once a week, for six weeks, with 3 consecutive doses.

The procedures for the test were as follows:

(1) The long diameter and short diameter of tumor were measured twice a week, with a vernier caliper, and the tumor volume (mm³) was calculated as =0.5×(tumor long diameter×tumor short diameter²).

(2) Relative inhibition rate of tumor TGI (%): TGI %=(1-T/C)×100%. T/C % is the relative proliferation rate of tumor (i.e. the percentage value of the tumor volume or tumor weight in the treatment group relative to the control group, at a certain time point). T and C are tumor volume (TV) or tumor weight (TW) of the treatment group and IgG1 control group at a specific time point, respectively.

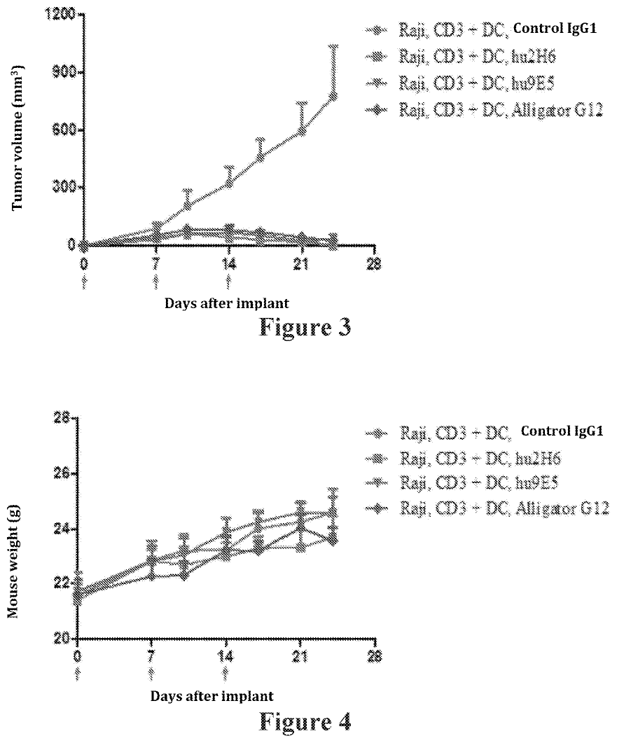

The results show that the humanized anti-CD40 antibodies hu2H6 and hu9E5 have very significant anti-tumor effects when compared to that of the IgG1 control. The tumor was almost completely eliminated on day 21 after administration; the anti-tumor effect was equivalent to or slightly better than that of the control antibody G12, as shown in FIG. 3 and FIG. 4.

### Example 12 Preparation of Anti-Cd40 Antibody Comprising Mutation(s) in Heavy Chain Constant Region

In this example, variants of the anti-CD40 antibody described above were prepared, which have mutation(s) in the heavy chain constant region.

In particular:
The amino acid at position 266 of hu2H6 heavy chain of SEQ ID NO: 17 was mutated from serine (S) to glutamic acid (E), to obtain mutant hu2H6-M;
The amino acid at position 266 of hu2H6 heavy chain of SEQ ID NO: 17 was mutated from serine (S) to glutamic acid (E), the amino acid at position 324 was mutated from asparagine (N) to serine (S), and the amino acid at position 327 was mutated from leucine (L) to phenylalanine (F), to obtain mutant hu2H6-SELFNS;
The amino acid at position 262 of hu9E5 heavy chain of SEQ ID NO: 19 was mutated from serine (S) to glutamic acid (E), to obtain mutant hu9E5-M;
The amino acid at position 262 of hu9E5 heavy chain of SEQ ID NO: 19 was mutated from serine (S) to glutamic acid (E), and the amino acid at position 323 was mutated from leucine (L) to phenylalanine (F), to obtain mutant hu9E5-SELF;
The amino acid at position 262 of hu9E5 heavy chain of SEQ ID NO: 19 was mutated from serine (S) to glutamic acid (E), the amino acid at position 320 was mutated from asparagine (N) to serine (S), and the amino acid at position 323 was mutated from leucine (L) to phenylalanine (F), to obtain mutant hu9E5-SELFNS.

The heavy chain sequences of hu2H6-M and hu2H6-SELFNS are shown in SEQ ID NOs: 61 and 62, and the light chain sequence is shown in SEQ ID NO: 18.

The heavy chain sequences of hu9E5-M, hu9E5-SELF, and hu9E5-SELFNS are shown in SEQ ID NO: 63, 64 and 67 and the light chain sequence is shown in SEQ ID NO: 20.

hu2H6-M HC:
(SEQ ID NO: 61)
QVQLVQSGAEVKKPGSSVKVSCKASGYAFSDYLIEWVRQAPGQGLEWM
GVINPGSGGSNYNEKIKDRVTLTADKSTSTAYMELSSLRSEDTAVYYC
ARGGGGFTYWGQGTLVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVEHEDPEVKFNWYVDGVEVHNAKT
KPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPGK;
hu2H6-SELFNS HC:
(SEQ ID NO: 62)
QVQLVQSGAEVKKPGSSVKVSCKASGYAFSDYLIEWVRQAPGQGLEWM
GVINPGSGGSNYNEKIKDRVTLTADKSTSTAYMELSSLRSEDTAVYYC
ARGGGGFTYWGQGTLVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVEHEDPEVKFNWYVDGVEVHNAKT
KPREEQYNSTYRVVSVLTVLHQDWLNGKEYKCKVSSKAFPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPGK;
hu9E5-M HC:
(SEQ ID NO: 63)
QVQLVQSGAEVKKPGASVKVSCKASGYILTTYWITWVRQAPGQGLEWM
GDIHPGSGSTKYNEKFKSRVTLTVDTSISTAYMELSRLRSEDTAVYYC
ARRDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPP
KPKDTLMISRTPEVTCVVVDVEHEDPEVKFNWYVDGVEVHNAKTKPRE
EQYNSTYRVVSVLTVLHQDWLNGKEYKCKVSNKAFPAPIEKTISKAKG
QPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYT
QKSLSLSPGK;
hu9E5-SELF HC:
(SEQ ID NO: 64)
QVQLVQSGAEVKKPGASVKVSCKASGYILTTYWITWVRQAPGQGLEWM
GDIHPGSGSTKYNEKFKSRVTLTVDTSISTAYMELSRLRSEDTAVYYC
ARRDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPP
KPKDTLMISRTPEVTCVVVDVEHEDPEVKFNWYVDGVEVHNAKTKPRE
EQYNSTYRVVSVLTVLHQDWLNGKEYKCKVSNKAFPAPIEKTISKAKG
QPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYT
QKSLSLSPGK;
hu9E5-SELFNS HC:
(SEQ ID NO: 67)
QVQLVQSGAEVKKPGASVKVSCKASGYILTTYWITWVRQAPGQGLEWM
GDIHPGSGSTKYNEKFKSRVTLTVDTSISTAYMELSRLRSEDTAVYYC
ARRDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPP
KPKDTLMISRTPEVTCVVVDVEHEDPEVKFNWYVDGVEVHNAKTKPRE
EQYNSTYRVVSVLTVLHQDWLNGKEYKCKVSSKAFPAPIEKTISKAKG
QPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYT
QKSLSLSPGK.

In addition, the amino acid K at the last position of SEQ ID NO: 61, 62, 63, 64 and 67 can be replaced with A. This mutation does not affect the activity of the antibody, but can improve the drugability of the antibody to a certain degree.

In addition, according to the variable region of another anti-CD40 antibody APX005S267E described in CN104918957A (i.e. amino acids at positions 1-120), antibody 005M was prepared as a positive control, and the amino acid sequence from positions 121 to 450 of the heavy chain of antibody 005M is the same as the amino acid sequence from positions 113 to 442 of heavy chain of antibody hu9E5-M. The specific sequences of 005M are as follows:

005M-HC:
(SEQ ID NO: 65)
QVQLVESGGGVVQPGRSLRLSCAASGFSFSSTYVCWVRQAPGKGLEWI
ACIYTGDGTNYSASWAKGRFTISKDSSKNTVYLQMNSLRAEDTAVYFC
ARPDITYGFAINFWGPGTLVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGG
PSVFLFPPKPKDTLMISRTPEVTCVVVDVEHEDPEVKFNWYVDGVEVH
NAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMH
EALHNHYTQKSLSLSPGK;
005M-LC:
(SEQ ID NO: 66)
DIQMTQSPSSLSASVGDRVTIKCQASQSISSRLAWYQQKPGKPPKLLI
YRASTLASGVPSRFSGSGSGTDFTLTISSLQPEDVATYYCQCTGYGIS
WPIGGGTKVEIKRTVAAPSVFIFPPSDEQLKSGTASVVCLLNNFYPRE
AKVQWKVDNALQSGNSQESVTEQDSKDSTYSLSSTLTLSKADYEKHKV
YACEVTHQGLSSPVTKSFNRGEC;

The amino acid at position 331 of 005M heavy chain of SEQ ID NO: 65 was mutated from leucine (L) to phenylalanine (F) to obtain mutant APX005-SELF; The amino acid at position 328 of 005M heavy chain of SEQ ID NO: 65 was mutated from asparagine (N) to serine (S), and the amino acid on position 331 was mutated from leucine (L) to phenylalanine (F) to obtain the mutant APX005-SELFNS.

APX005-SELF HC (L331F):
(SEQ ID NO: 68)
QVQLVESGGGVVQPGRSLRLSCAASGFSFSSTYVCWVRQAPGKGLEWI
ACIYTGDGTNYSASWAKGRFTISKDSSKNTVYLQMNSLRAEDTAVYFC
ARPDITYGFAINFWGPGTLVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGG
PSVFLFPPKPKDTLMISRTPEVTCVVVDVEHEDPEVKFNWYVDGVEVH
NAKTKPREEQYNSTYRVVSVLTVLHQDWLNGKEYKCKVSNKAFPAPIE
KTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMH
EALHNHYTQKSLSLSPGK;
APX005-SELFNS HC(L331F,N328S):
(SEQ ID NO: 69)
QVQLVESGGGVVQPGRSLRLSCAASGFSFSSTYVCWVRQAPGKGLEWI
ACIYTGDGTNYSASWAKGRFTISKDSSKNTVYLQMNSLRAEDTAVYFC
ARPDITYGFAINFWGPGTLVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGG
PSVFLFPPKPKDTLMISRTPEVTCVVVDVEHEDPEVKFNWYVDGVEVH
NAKTKPREEQYNSTYRVVSVLTVLHQDWLNGKEYKCKVSSKAFPAPIE
KTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMH
EALHNHYTQKSLSLSPGK;

The antibodies hu2H6-M, hu2H6-SELFNS, hu9E5-M, hu9E5-SELF, hu9E5-SELFNS and 005M, APX005-SELF and APX005-SELFNS were prepared and confirmed by sequencing.

### Example 13 Test of Anti-Cd40 Antibody Comprising Mutation(s) in Heavy Chain Constant Region to Activate Dc Cells

PBMCs were isolated from the peripheral blood of normal human subject, and then monocytes were sorted using CD14 MACS beads. RPMI 1640 medium comprising 25 ng/mL IL-4 and 50 ng/mL GM-CSF was added for cultivation for 6 days to induce MoDC cells (dendritic cells derived from monocytes).

Cells were collected after 6 days, 1×10⁵ cells were taken, and stained with CD209-PE, CD1a-PerCP/Cy5.5 and CD14-PE/Cy7 for analyzing whether MoDC has been successfully induced by FACS (the above operations are routine operations in the art). The successfully induced DCs were collected, each antibody to be tested and control antibody were added, and the corresponding concentration dilution gradients were set up to obtain antibody gradients: 0.01 nM, 0.16 nM, 0.8 nM, 4 nM, 20 nM, 100 nM. After cultivating for 48 hours, the cells were collected and stained using CD86 and HLA-DR staining, and data was collected by FACS. Both APX005M-SELFNS and 2H6-SELFNS showed stronger agonist activity than that of Alligator control antibody G12, and activated the activation molecule CD86 on the surface of DC cells in a dose-dependent way (See FIG. 6).

### Example 14 Test of Anti-Cd40 Antibody Comprising Mutation(s) in Heavy Chain Constant Region for Activation of Dc Cells to Produce Cytokines

PBMCs were isolated from the peripheral blood of normal human subject, and then monocytes were sorted using CD14 MACS beads. RPMI 1640 medium comprising 25 ng/mL IL-4 and 50 ng/mL GM-CSF was added for cultivation for 6 days to induce MoDC cells (dendritic cells derived from monocytes). Cells were collected after 6 days, 1×10⁵ cells were taken, and stained with CD209-PE, CD1a-PerCP/Cy5.5 and CD14-PE/Cy7 for analyzing whether MoDC has been successfully induced by FACS (the above operations are routine operations in the art). The successfully induced DCs were collected, each antibody to be tested and control antibody were added, and the corresponding concentration dilution gradients were set up to obtain antibody gradients: 0.01 nM, 0.16 nM, 0.8 nM, 4 nM, 20 nM, 100 nM. After cultivating for 48 hours, the supernatant was collected and the content of IL-12 p40 was detected by ELISA.

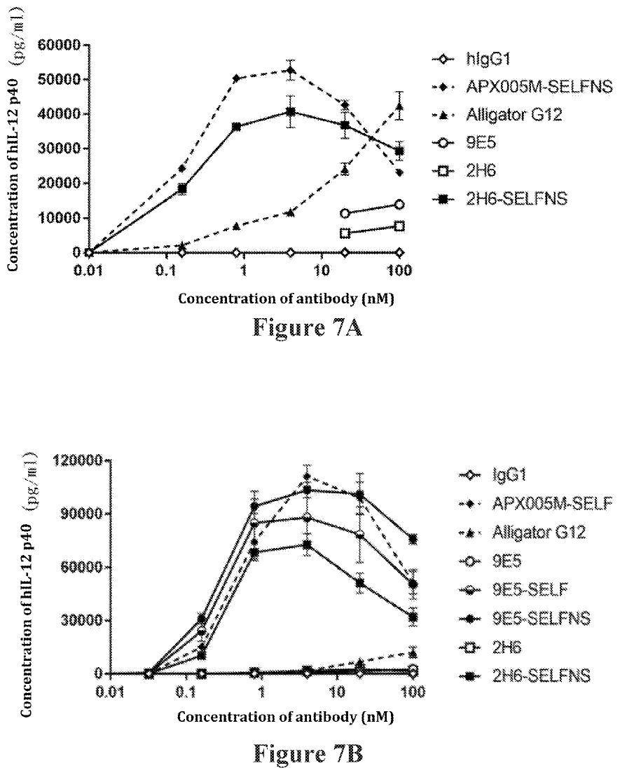

APX005M-SELFNS, APX005M-SELF, 2H6-SELFNS and 9E5-SELFNS all showed stronger agonist activity than that of Alligator control antibody G12, and promoted the secretion of cytokine IL-12 p40 from DC cells in a dose-dependent way. The results are shown in FIG. 7A, FIG. 7B and Table 12, Table 13.

TABLE 12
Results of anti-CD40 antibody promoting the
secretion of cytokine IL-12 p40 from DC cells

| Concentration of | | | |
|---|---|---|---|
| antibody (nM) | hIgG1 | APX005M-SELFNS | Alligator G12 |
| 100 | 65.205 ± 14.145 | 23129.857 ± 1123.371 | 42475.280 ± 4060.051 |
| 20 | 76.817 ± 33.036 | 42648.367 ± 1338.211 | 24147.463 ± 1685.812 |
| 4 | 35.016 ± 6.301 | 52787.687 ± 2854.792 | 11789.560 ± 375.848 |
| 0.8 | 34.952 ± 4.832 | 50373.157 ± 518.778 | 7762.352 ± 189.066 |
| 0.16 | 20.899 ± 2.246 | 24311.430 ± 228.650 | 2110.560 ± 87.567 |
| 0.01 | 35.562 ± 12.971 | 35.562 ± 12.971 | 35.562 ± 12.971 |

| Concentration of | | | |
|---|---|---|---|
| antibody (nM) | 9E5 | 2H6 | 2H6-SELFNS |
| 100 | 13998.987 ± 349.462 | 7668.062 ± 773.460 | 29300.717 ± 2741.181 |
| 20 | 11397.870 ± 428.991 | 5633.816 ± 335.383 | 36753.370 ± 3812.485 |
| 4 | / | / | 40700.567 ± 4621.792 |
| 0.8 | / | / | 36329.460 ± 790.604 |
| 0.16 | / | / | 18411.147 ± 1639.285 |
| 0.01 | / | / | 35.562 ± 12.971 |

(Note:
“/” means that the concentration was not tested).

TABLE 13
Results of anti-CD40 antibody promoting the secretion of cytokine IL-12 p40 from DC cells

| Con. of | | | | | |
|---|---|---|---|---|---|
| antibody nM | IgG1 | APX005M-SELF | Alligator G12 | 9E5 | 9E5-SELF |
| 100 | 0.000 | 51366.137 ± 6387.822 | 12099.219 ± 3111.489 | 2564.309 ± 605.549 | 50421.003 ± 8453.659 |
| 20 | 0.000 | 99039.430 ± 8959.730 | 6798.054 ± 1207.029 | 1320.558 ± 328.965 | 78407.500 ± 15600.894 |
| 4 | 0.000 | 111253.733 ± 6263.173 | 1604.377± 533.314 | 889.822 ± 237.943 | 88086.533 ± 7487.812 |
| 0.8 | 0.000 | 74423.800 ± 9486.879 | 698.189 ± 301.846 | 787.522 ± 278.889 | 84902.653 ± 7840.563 |
| 0.16 | 0.000 | 15199.523 ± 1874.331 | 0.000 | 0.000 | 24249.347 ± 5744.800 |
| 0.032 | 0.000 | 0.000 | 0.000 | 0.000 | 211.413 ± 105.920 |
| 0.0064 | 0.000 | 0.000 | 0.000 | 0.000 | 0.000 |

| Con. of | | | |
|---|---|---|---|
| antibody nM | 9E5-SELFNS | 2H6 | 2H6-SELFNS |
| 100 | 75819.840 ± 2768.239 | 1426.910 ± 244.872 | 32000.920 ± 5042.054 |
| 20 | 101297.360 ± 1534.936 | 2508.316 ± 627.554 | 51167.883 ± 5724.671 |
| 4 | 103433.200 ± 4360.661 | 1597.402 ± 568.304 | 72797.640 ± 6296.468 |
| 0.8 | 94355.880 ± 4121.238 | 564.707 ± 194.116 | 68460.980 ± 4612.750 |
| 0.16 | 31196.040 ± 2942.471 | 0.000 | 10264.087 ± 2045.021 |
| 0.032 | 642.149 ± 247.146 | 0.000 | 0.000 |
| 0.0064 | 0.000 | 0.000 | 0.000 |

### Example 15 Inhibition of Mouse Tumor Growth by Anti-Cd40 Antibody Comprising Mutation(s) in Heavy Chain Constant Region

In this example, the anti-tumor effect and safety of administration of CD40 antibody were evaluated by the size of tumor and the weight of mice on an MC38 tumor model of humanized hFcγR/hCD40 C57BL/6 mouse.

The method for cultivating and preparing MC38 cells: MC38 mouse colon cancer cell line was cultivated in DMEM (comprising 10% FBS, 1% penicillin-streptomycin, 1 mM sodium pyruvate and 10 mM HEPES), and the cells were proliferated to reach a density of 80%-90% in the culture plate. Trypsin-EDTA (0.25%) was added and incubated at 37° C. for 3 to 5 minutes for digestion, and medium comprising 10% FBS was used to terminate the reaction. The cells were centrifuged and washed for twice with PBS, and finally re-suspended in PBS to prepare a single cell suspension, and the cell density was adjusted to 10⁷ cells/mL for later use.

The method for establishing MC38 tumor model: the MC38 single cell suspension prepared above (2×10⁶ MC38 cells, 200 μL) was used to subcutaneously inoculate 32 humanized hFcγR/hCD40 C57BL/6 mice (provided by LI Fubin team, Department of Medicine, Shanghai Jiaotong University, kept at SPF level) at right flank on day 7. When the average tumor volume in mice reached about 55 mm³, they were randomly divided into 4 groups with 8 mice in each group.

After grouping, a single dose of anti-CD40 antibody was administered intraperitoneally according to the regimen shown in Table 12. The tumor volume and the body weight was measured twice a week, and the data was recorded. Among them, the control IgG, hu9E5, and hu9E5-M were provided by Shanghai Hengrui Pharmaceutical Co., Ltd. and diluted with PBS to obtain a final concentration 0.3 mg/mL.

Indicators for evaluating anti-tumor activity of antibody:

1) The tumor volume of the mice was measured continuously after the subject mice were divided into groups, and the size of tumor volume was used as an indicator to evaluate the anti-tumor activity of the antibody to be tested. The formula to calculate tumor volume (TV) is as follows:

TV=0.5×Lₛₕₒᵣₜ×Lₛₕₒᵣₜ×L_{long},

where Lₛₕₒᵣₜ is the shortest diameter of tumor, and L_{long} is the longest diameter of tumor.

2) T/C % is the relative tumor proliferation rate, i.e., the percentage value of tumor volume in the treatment group relative to that in the control group, at a certain time point, which is calculated as follows:

T/C%=(T−T₀)/(C−C₀)×100

where T and C refer to the tumor volume at the end of the test; To, Co refer to the tumor volume at the beginning of the test.

3) Relative inhibition rate of tumor TGI (%)=(1−T/C)×100%.

Data expression and statistical processing: All data were analyzed by GraphPad Prism 5.0 software. The data are expressed as Mean±standard deviation, and one-way ANOVA analysis was used between groups. P<0.05 indicates that the difference is statistically significant.

TABLE 14
Test grouping and dosing regimen

| | | | | | Adminis- |
|---|---|---|---|---|---|
| | | | Dose | Dosing | tration |
| Test grouping | Number | Grouping | (mg/kg) | regimen | route |
| Group 1 | 8 | Control | 3 | D 0, D 3, | ip |
| | | (Control | | D 6 | |
| | | IgG) | | | |
| Group 2 | 8 | hu9E5 | 3 | D 0, D 3, | ip |
| | | | | D 6 | |
| Group 3 | 8 | hu9E5-M | 3 | D 0, D 3, | ip |
| | | | | D 6 | |
| Group 4 | 8 | 005M | 3 | D 0, D 3, | ip |
| | | | | D 6 | |

(Note:
once every three days, and for 3 times in total; ip: intraperitoneal injection).

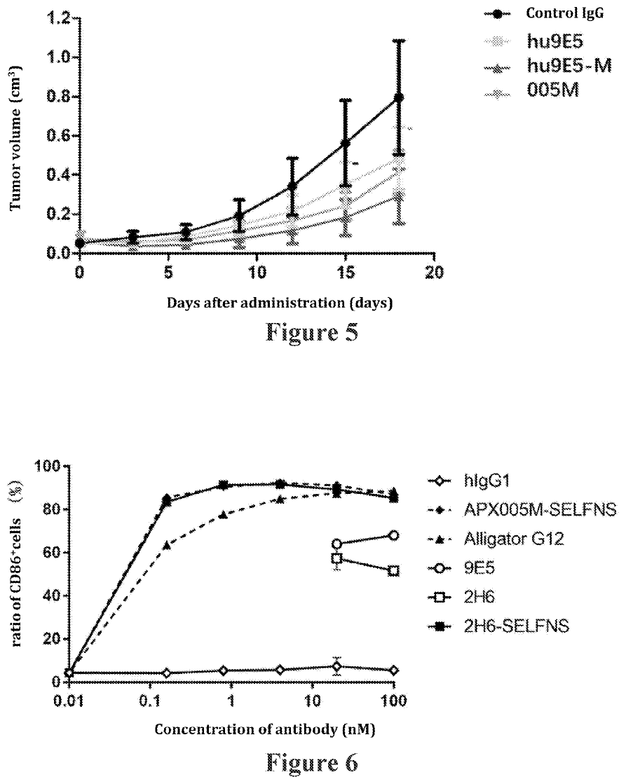

The in vivo activity results for each group of antibodies in hFcγR/hCD40Tg mouse MC38 tumor model can be judged by the change in tumor volume. After the control antibody and test antibodies were administered on day 0, day 3, and day 6, the growth of mouse tumor volume was inhibited in hu9E5 group, hu9E5-M group and 005M group, when compared with that in control group (control IgG). The relative tumor inhibition rates were 42.0%, 68.9%, and 53.8%, respectively. Hu9E5 has certain anti-tumor activity (p>0.05), hu9E5-M and 005M have strong anti-tumor activity (p<0.05), and hu9E5-M has more beneficial effects than that of 005M, as shown in Table 15 and FIG. 5.

TABLE 15
Tumor volume (cm)³

| | | | | Inhibition |
|---|---|---|---|---|
| | | | | rate of |
| | | tumor volume | | tumor |
| | | (Mean±SEM) | | volume (%) |
| | Grouping | D0 | D18 | D18 |
| Group 1 | Control | 0.052 ± 0.015 | 0.794 ± 0.29 | / |
| | (Control | | | |
| | IgG) | | | |
| Group 2 | hu9E5 | 0.053 ± 0.016 | 0.483 ± 0.159 | 42.0% |
| Group 3 | hu9E5-M | 0.060 ± 0.022 | 0.291 ± 0.139 | 68.9%* |
| Group 4 | 005M | 0.069 ± 0.03 | 0.412 ± 0.105 | 53.8%* |

(Note:
*p < 0.05 or lower, indicating statistical significance).

The results show that the amino acid mutation (from S to E) at position 266 corresponding to SEQ ID NO: 17 or at position 262 corresponding to SEQ ID NO: 19 can significantly improve the in vivo anti-tumor effect of the anti-CD40 antibodies of present application.

Although the specific embodiments of the present invention are described above, those skilled in the art should understand that these embodiments are only for exemplary purpose, various changes or modifications can be made to these embodiments without departing from the principle and essence of the present invention. Therefore, the protection scope of the present invention is defined by the appended claims.

1. An anti-CD40 antibody or antigen-binding fragment thereof, which comprises mutation(s) in heavy chain constant region, wherein:
1) a light chain variable region of the antibody comprises LCDR1, LCDR2 and LCDR3 as shown in SEQ ID NO: 6, SEQ ID NO: 7 and SEQ ID NO: 8, respectively; and
a heavy chain variable region of the antibody comprises HCDR1, HCDR2 and HCDR3 as shown in SEQ ID NO: 3, SEQ ID NO: 4 and SEQ ID NO: 5, respectively;
or,
2) a light chain variable region of the antibody comprises LCDR1, LCDR2 and LCDR3 as shown in SEQ ID NO: 14, SEQ ID NO: 15 and SEQ ID NO: 16, respectively; and
a heavy chain variable region of the antibody comprises HCDR1, HCDR2 and HCDR3 as shown in SEQ ID NO: 11, SEQ ID NO: 12 and SEQ ID NO: 13, respectively;
or,
3) a light chain variable region of the antibody comprises LCDR1, LCDR2 and LCDR3 as shown in SEQ ID NO: 42, SEQ ID NO: 43 and SEQ ID NO: 44, respectively; and
a heavy chain variable region of the antibody comprises HCDR1, HCDR2 and HCDR3 as shown in SEQ ID NO: 39, SEQ ID NO: 40 and SEQ ID NO: 41, respectively;
or,
4) a light chain variable region of the antibody comprises LCDR1, LCDR2 and LCDR3 as shown in SEQ ID NO: 50, SEQ ID NO: 51 and SEQ ID NO: 52, respectively; and
a heavy chain variable region of the antibody comprises HCDR1, HCDR2 and HCDR as shown in SEQ ID NO: 47, SEQ ID NO: 48 and SEQ ID NO: 49, respectively;
or,
5) a light chain variable region of the antibody comprises LCDR1, LCDR2 and LCDR3 as shown in SEQ ID NO: 58, SEQ ID NO: 59 and SEQ ID NO: 60, respectively; and
a heavy chain variable region of the antibody comprises HCDR1, HCDR2 and HCDR3 as shown in SEQ ID NO: 55, SEQ ID NO: 56 and SEQ ID NO: 57, respectively.

2. The anti-CD40 antibody or antigen-binding fragment thereof according to claim 1, wherein the antibody or antigen-binding fragment thereof is a murine antibody, a chimeric antibody, a humanized antibody, a human antibody or an antigen-binding fragment thereof.

3. The anti-CD40 antibody or antigen-binding fragment thereof according to claim 2, wherein the variable region amino acid sequences of the murine antibody or the chimeric antibody are selected from the group consisting of:
1) a heavy chain variable region as shown in SEQ ID NO: 1, and a light chain variable region as shown in SEQ ID NO: 2;
2) a heavy chain variable region as shown in SEQ ID NO: 9, and a light chain variable region as shown in SEQ ID NO: 10;
3) a heavy chain variable region as shown in SEQ ID NO: 37, and a light chain variable region as shown in SEQ ID NO: 38;
4) a heavy chain variable region as shown in SEQ ID NO: 45, and a light chain variable region as shown in SEQ ID NO: 46; and
5) a heavy chain variable region as shown in SEQ ID NO: 53, and a light chain variable region as shown in SEQ ID NO: 54.

4. The anti-CD40 antibody or antigen-binding fragment thereof according to claim 2, wherein the heavy chain variable region of the humanized antibody further comprises:
heavy chain FRs of human IgG1, IgG2, IgG3 or IgG4 or variants thereof.

5. The anti-CD40 antibody or antigen-binding fragment thereof according to claim 2, wherein:
the light chain FRs in the light chain variable region of the humanized antibody are derived from:
a human germline light chain IGkV1-33 sequence as shown in SEQ ID NO: 22, or
a human germline light chain IGkV2-28 sequence as shown in SEQ ID NO: 24;
the heavy chain FRs in the heavy chain variable region of the humanized antibody are derived from:
a human germline heavy chain IGHV1-69 sequence as shown in SEQ ID NO: 21, or
a human germline heavy chain IGHV1-2 sequence as shown in SEQ ID NO: 23.

6. The anti-CD40 antibody or antigen-binding fragment thereof according to claim 5, wherein:
the humanized antibody light chain is as shown in SEQ ID NO: 18 or SEQ ID NO: 20 or variant thereof; the variant has 0 to 10 amino acid mutation(s) in the light chain variable region; and/or
before mutation of the heavy chain constant region, the humanized antibody heavy chain is as shown in SEQ ID NO: 17 or SEQ ID NO: 19 or variant thereof; the variant has 0 to 10 amino acid mutation(s) in the heavy chain variable region.

7. The anti-CD40 antibody or antigen-binding fragment thereof according to claim 6, wherein:
the heavy chain variable region is as shown in one of SEQ ID NOs: 25-30 or a variant thereof,
the light chain variable region is as shown in one of SEQ ID NOs: 31-36 ora variant thereof.

8. The anti-CD40 antibody or antigen-binding fragment thereof according to claim 1-7, wherein the heavy chain comprises mutation(s) of amino acid residue(s) at position(s) selected from the group consisting of:
1) position 262 corresponding to SEQ ID NO: 19 or position 266 corresponding to SEQ ID NO: 17; and/or
2) position 320 corresponding to SEQ ID NO: 19 or position 324 corresponding to SEQ ID NO: 17; and/or
3) position 323 corresponding to SEQ ID NO: 19 or position 327 corresponding to SEQ ID NO: 17.

9. The anti-CD40 antibody or antigen-binding fragment thereof according to claim 8, wherein the heavy chain comprises mutation(s) selected from the group consisting of:
1) the amino acid residue at position 262 corresponding to SEQ ID NO: 19 or at position 266 corresponding to SEQ ID NO: 17 is mutated to glutamic acid; and/or
2) the amino acid residue at position 320 corresponding to SEQ ID NO: 19 or at position 324 corresponding to SEQ ID NO: 17 is mutated to serine; and/or
3) the amino acid residue at position 323 corresponding to SEQ ID NO: 19 or at position 327 corresponding to SEQ ID NO: 17 is mutated to phenylalanine.

10. The anti-CD40 antibody or antigen-binding fragment thereof according to claim 9, wherein the heavy chain comprises mutation(s) selected from the following, or the combination thereof:
1) 262E corresponding to SEQ ID NO: 19;
2) 262E and 323F corresponding to SEQ ID NO: 19;
3) 262E, 320S and 323F corresponding to SEQ ID NO: 19;
4) 266E corresponding to SEQ ID NO: 17; or
5) 266E, 324S and 327F corresponding to SEQ ID NO: 17.

11. The anti-CD40 antibody or antigen-binding fragment thereof according to claim 1, wherein the humanized antibody comprises:
a heavy chain as shown in SEQ ID NO: 61 or 62, and a light chain as shown in SEQ ID NO: 18; or the humanized antibody comprises:
a heavy chain as shown in SEQ ID NO: 63, 64 or 67, and
a light chain as shown in SEQ ID NO: 20.

12. The anti-CD40 antibody or antigen-binding fragment thereof according to claim 1, wherein the amino acid residue at the carboxyl terminus of the heavy chain is mutated to alanine residue.

13. A single-chain antibody, which comprises:
the light chain variable region and the heavy chain variable region as defined in claim 1.

14. An antibody-drug conjugate, wherein the antibody comprises the light chain variable region and the heavy chain variable region as defined in claim 1.

15. A nucleic acid molecule encoding the anti-CD40 antibody or the antigen-binding fragment of claim 1.

16. A vector comprising the nucleic acid molecule of claim 15.

17. A host cell comprising or expressing the vector of claim 16.

18. (canceled)

19. A pharmaceutical composition comprising:
the anti-CD40 antibody or antigen-binding fragment thereof of claim 1, and
a pharmaceutically acceptable excipient, diluent or carrier.

20. (canceled)

21. (canceled)

22. A method for preventing or treating CD40-mediated or CD40L-mediated disease, the method comprising contacting a subject with a therapeutically effective amount of the anti-CD40 antibody or antigen-binding fragment thereof of claim 1.

23. A method for improving symptom(s) of an autoimmune disease or an inflammatory disease, the method comprising contacting a subject with a prophylactically effective amount or a therapeutically effective amount of the anti-CD40 antibody or antigen-binding fragment thereof of claim 1.
